(19) United States
(12) Patent Application Publication
(10) Pub. No.: US 2025/0135050 A1
(43) Pub. Date: May 1, 2025

(54) IODIDE NANOPARTICLES AND COMPOSITIONS OF METHODS OF USE THEREOF

(71) Applicants: University of Georgia Research Foundation, Inc., Athens, GA (US); The University of North Carolina At Chapel Hill, Chapel Hill, NC (US)

(72) Inventors: Jin Xie, Watkinsville, GA (US); Zibo Li, Chapel Hill, NC (US)

(21) Appl. No.: 18/690,230

(22) PCT Filed: Sep. 7, 2022
(86) PCT No.: PCT/US2022/076036

§ 371 Date: Mar. 7, 2024

Related U.S. Application Data
(60) Provisional application No. 63/241,262, filed on Sep. 7, 2021.

(51) Int. Cl. A61K 51/12; A61K 41/00; A61K 45/06; A61K 47/69; A61P 35/00

(52) U.S. Cl. CPC A61K 51/1244; A61K 41/0038; A61K 45/06; A61K 47/6929; A61P 35/00; A61K 2121/00; A61K 2123/00

(57) ABSTRACT

Iodide nanoparticles are provided. The nanoparticles can be potassium iodide (KI) nanoparticles, and can be formed of, a radioisotope, preferably ¹³¹I or ¹²⁵I. The nanoparticles can include a coating that can increase the half-life of the particles, serve as a platform for attachment or entrapment of targeting moieties and/or additional active agents, or a combination thereof. Methods of use are also provided. For example, a method of treating a subject for cancer can including sensitizing the subject to radiation therapy by administrating the iodide nanoparticles to the subject in combination with one or more doses of radiation therapy. Methods of treating or imaging a subject can also include administering the subject an effective amount iodide nanoparticles, without the nanoparticles serving as radiosensitizer. In such methods, the nanoparticles typically include a radioisotope or an anticancer active agent, for example ¹³¹I, ¹²⁵I, ¹²⁴I, or ¹²³I, or a chemotherapeutic agent.

### CROSS-REFERENCE TO RELATED APPLICATION

This application claims the benefit of and priority to U.S. Ser. No. 63/241,262 filed Sep. 7, 2021, and which is incorporated by reference in its entirety.

### STATEMENT REGARDING FEDERALLY SPONSORED RESEARCH OR DEVELOPMENT

This invention was made with government support under R01 EB022596 awarded by the National Institutes of Health. The government has certain rights in the invention. (37 CFR 401.14 f (4)).

### FIELD OF THE INVENTION

The invention is generally directed to particle compositions and methods of use thereof, particularly for radiotherapy.

### BACKGROUND OF THE INVENTION

Breast cancer is the most commonly diagnosed cancer among women and the second most common cause of cancer mortality among women (Jemal, et al., CA: a cancer journal for clinicians, 60 (5), 277-300 (2010)). Radiotherapy (RT) (also radiation therapy) plays an integral role in breast cancer therapy. RT can be applied after lumpectomy or mastectomy to reduce tumor recurrence (Baskar, et al., International journal of medical sciences, 9 (3), 193 (2012), Haussmann, et al., Radiat Oncol, 15 (1), 71 (2020)), or given in a neoadjuvant setting for managing locally advanced breast cancer (Poleszczuk, et al., Breast Cancer Res, 19 (1), 75 (2017)). However, the dose and efficacy of RT is limited by normal tissue toxicity. To improve treatment outcomes, radiosensitizers, agents that can enhance RT efficacy at the same irradiation dose, may be applied during or after radiation. Conventional radiosensitizers are chemotherapeutics such as paclitaxel, cyclophosphamide, and fluorouacil (Candelaria, et al., Radiation Oncology, 1 (1), 15 (2006)). More recently, high atomic number or high-Z nanoparticles (NPs) that are made of gold (Cui, et al., Radiother Oncol, 124 (3), 344-356 (2017)), hafnium oxide (Maggiorella, et al., Future Oncol, 8 (9), 1167-81 (2012)), and metal-organic frameworks (MOFs) (Meng, et al., Theranostics, 8 (16), 4332-4344 (2018)) are tested as radiosensitizers and show promising results. High-Z NPs afford large cross-section for high-energy photons thus increasing energy deposition in tumors. This results in elevated production of toxic reactive oxygen species (ROS) that kill cancer cells (Su, et al., Cancer biology & medicine, 11 (2), 86 (2014), Porcel, et al., Nanotechnology, 21 (8), 085103 (2010), Chithrani, et al., Radiation research, 173 (6), 719-728 (2010)). However, most high-Z NPs have relatively large sizes, restraining their ability to diffuse within the dense tumor tissues and their uptake by cancer cells (Waite, et al., Critical Reviews™ in Biomedical Engineering, 40 (1) (2012)). This restriction may negatively affect radiosensitizing effects as ROS have a short effective range (10-400 nm) and limited ability to penetrate biological barriers such as the plasma membrane (Schmitt, et al., Biochimica et Biophysica Acta (BBA)-Bioenergetics, 1837 (6), 835-848 (2014)).

In addition to high-Z NPs made of metals or metal oxides, iodine compounds have also shown radiosensitizing benefits. As early as in the 50s and 60s, it was found that iodine compounds such as iodoacetate, iodoacetamide, and potassium iodide could enhance radiative lethality against bacteria and mammalian cells (Dean & Alexander, Progr. Biochem. Pharmacol., 1 (1965), Bridges, Radiation research, 16 (3), 232-242 (1962), Dean & Alexander, Nature, 196 (4861), 1324-1326 (1962)). Recent studies show that iodinated computed tomography (CT) contrast agents, such as Iomeprol, Iopromide and Iomeron (Tamura, et al., Scientific reports, 7, 43667 (2017), Wang, et al., European journal of radiology, 92, 72-77 (2017), Obeid, et al., Journal of Cerebral Blood Flow & Metabolism, 34 (4), 638-645 (2014)) can increase radiation-induced cancer cell death. This discovery has led to clinical studies examining iodine-enhanced RT for brain tumor (Meng, et al., Theranostics, 8 (16), 4332-4344 (2018)). However, iodinated contrast agents have very short circulation half-lives and low tumor retention (Meng, et al., Theranostics, 8 (16), 4332-4344 (2018)). While measures such as infusion can be taken to improve tumor concentration of iodine at the time of irradiation, the efficacy is still limited by low accumulation of iodine inside cancer cells (Cormode, et al., Contrast media & molecular imaging, 9 (1), 37-52 (2014), Silva, et al., Radiologia brasileira, 51 (4), 236-241 (2018)).

Therefore, there remains a need for improved iodine-based therapeutics.

Thus, it is an object of the invention to provide improved iodine-based compositions and methods of use thereof.

### SUMMARY OF THE INVENTION

Iodide nanoparticles are provided. The nanoparticles are typically formed of ions from iodine and an alkali metal or alkaline earth metal, such as potassium, lithium, sodium, rubidium, cesium, magnesium, or calcium. In preferred embodiments, the nanoparticles are potassium iodide (KI) nanoparticles. The particles can be formed of, or otherwise include, a radioisotope, preferably ¹³¹I, ¹²⁵I, ¹²³I, or ¹²⁴I. The nanoparticle can be, for example, cubic. In some embodiments, the nanoparticles are between about 15 nm and about 800 nm, or between about 20 nm and about 500 nm, or between about 50 nm and about 350 nm, or about 50 nm to about 125 nm, or any subrange or specific integer there between, such as about 80 nm. In preferred embodiments, the particle can be transport across a cell's plasma membrane by the sodium/iodide symporter (NIS).

The nanoparticles can include a coating, also referred a layer, which is typically external to the salt core of the particles. The coating can be formed from one or more polymers, peptides, proteins, lipids, silica, metal oxides, or a combination thereof. In particular embodiments, the coating includes poly(maleic anhydride alt-1-octadecene). The coating can increase the half-life of the particles, serve as a platform for attachment or entrapment of targeting moieties and/or additional active agents, or a combination thereof. The thickness of the coating can be tailored to control the half-life, release of the potassium iodide core and/or entrapped active agents, or a combination thereof. For example, the coating can have a thickness of 1 nm to 200 nm, or 10 nm to 100 nm, or 25 nm to 75 nm inclusive, or any subrange or specific integer therebetween, for example about 50 nm.

The targeting moiety typically enhances accumulation or retention of the particles at specific cell type(s) or tissue(s). The targeting moiety may target a cell surface or transmembrane protein, a cell specific marker, a tumor associated or cancer antigen, etc. A particular target is the sodium/iodide symporter (NIS). The targeting moiety can be a ligand, receptor, antibody, or aptamer, which can optionally be conjugated to a component of the coating. Active agents can be, for example, therapeutic, nutritional, diagnostic, or prophylactic agents. Exemplary therapeutic agents are chemotherapeutic agents. In some embodiments, the active agent is entrapped in a matrix formed by the coating.

Pharmaceutical compositions including a plurality of the nanoparticles are also provided, and can be administered to subjects in need thereof. In some embodiments, the average hydrodynamic size of the nanoparticles in the pharmaceutical composition is between about 15 nm and about 800 nm, or between about 20 nm and about 500 nm, or between about 50 nm and about 350 nm, or about 50 nm to about 125 nm, or any subrange or specific integer there between +5%, 10%, 15%, 20%, or 25%. In some embodiments, the nanoparticles are monodisperse.

Methods of making iodide particles, most particularly potassium iodide nanoparticles, are also provided. For example, some such methods include reacting potassium oleate with I2 in octadecene in the presence of oleylamine, and optionally, but preferably adding a coating such as poly(maleic anhydride alt-1-octadecene) to the KI core.

Methods of use are also provided. For example, a method of sensitizing a subject to radiation therapy can include administering to a subject in need thereof a pharmaceutical composition having an effective amount of the iodide nanoparticle.

A method of treating a subject for cancer can including sensitizing the subject to radiation therapy by administering the subject a combination of an iodide particle composition and one or more doses of radiation therapy. The radiation therapy is preferably ionizing radiation therapy, however, other forms are also contemplated. In some embodiments, the pharmaceutical composition enhances the treatment of the cancer compared to administration of the radiation alone. The cancer can be a radiosensitive cancer or a radioresistant cancer. Preferably, the same dose of radiation is more effective than when administered in the absence of the pharmaceutical composition, a lower dose of radiation has the same effectiveness as a higher dose when administered in the absence of the pharmaceutical composition, or a combination thereof. Typically, the dose of radiation is administered contemporaneously or after administration of the pharmaceutical composition, for example, 0 hour to 48 hours, or 0.5 hour to 24 hours, or 1 hour to 12 hours, or 1 hour to 6 hours, or 2 hours to 6 hours, or any subrange or specific integer or fraction thereof therebetween, for example 0 (i.e., contemporaneously with), or 0.25, 0.5, 0.75, 1, 2, 3, 4, or 5 hours after administration of the pharmaceutical composition. In some embodiments, the method includes 1, 2, 3, 4, 5, 6, 7, 8, 9, 10, or more rounds of administration of the pharmaceutical composition followed by administration of the dose of radiation. In some embodiments, the particles are only re-administered after 2, 3, 4, 5, or more dose of radiation.

Methods of treating a subject for cancer including administering the subject an effective amount a pharmaceutical composition including iodide nanoparticles, without the nanoparticles serving as radiosensitizer (i.e., an adjunct to secondary administration of radiation therapy) are also provided. In such methods, the nanoparticles typically include a radioisotope or an anti-cancer active agent, for example ¹³¹I or ¹²⁵I, or a chemotherapeutic agent. Particles harboring a radioisotope, also referred to herein as “hot” particles, can serve a radiotherapeutic function without the need for a second source of radiation, though may nonetheless optionally be combined with one.

In some embodiments, the cancer is a vascular, bone, muscle, bladder, brain, breast, cervical, colo-rectal, esophageal, kidney, liver, lung, nasopharangeal, pancreatic, prostate, skin, stomach, uterine, germ cell cancer, or metastasis thereof, or any combination thereof. In preferred embodiments, the cancer cells express the Na⁺/I⁻ symporter (NIS). In some embodiments, the cancer is a breast cancer or a thyroid cancer or a metastasis of the breast or thyroid cancer, or a combination thereof. Methods of administration are also provided and may be, e.g., systemic or local. In particularly embodiments, the pharmaceutical composition is injected or infused locally to the site in need of treatment, e.g., a tumor.

The disclosed compositions can also be used for imaging, e.g., by delivering radioiodine to tumors for imaging. Such methods typically include administering a subject an effective amount of iodide nanoparticles typically including a radioisotope, for example ¹²³I or ¹²⁴I, and then imaging the subject using an imaging device such as a PET and or SPECT system. Such methods can be used as part of diagnostic or prognostic method, including for diseases such as cancer. As with the methods of treatment, administration may be systemic or local to the site of interest.

### BRIEF DESCRIPTION OF THE DRAWINGS

FIG. 1A is transmission electron microscope (TEM) images of as-synthesized potassium iodide (KI) NPs. Scale bar, 500 nm. Insert scale bar, 100 nm. FIG. 1B is a bar graph showing dynamic light scattering (DLS) results of KI NPs in hexane. FIG. 1C is a histogram showing DLS characterization of KI NPs of different sizes. FIG. 1D is an x-ray powder diffraction (XRD) spectra of KI NPs and KI (PDF #04-005-6718). FIG. 1E is an energy-dispersive x-ray (EDS) spectrum and element weight percentages of KI NPs. FIG. 1F is a schematic representation of PMAO-KI NP synthesis, and surface coating of KI particles. KI NPs were synthesized in octadecene and then coated with a layer of PMAO to prevent fast degradation and I⁻ release.

FIG. 2A is TEM images of PMAO-KI NPs. Scale bars, Scale bar, 500 nm. Insert scale bar, 20 nm. FIG. 2B is a Fourier-transform infrared spectroscopy (FTIR) spectra of PMAO-KI NPs and PMAO polymer. FIG. 2C is a spectra showing the Zeta potential of PMAO-KI NPs. Due to surface carboxyl groups, PMAO-KI NPs carry a negative surface charge. FIG. 2D is a bar graph showing the results of DLS of PMAO-KI NPs (in PBS) and uncoated KI NPs (in hexane). FIG. 2E is a line graph showing iodide release, measured in PBS by iodide-selective electrode. FIG. 2F is a line graph showing hydroxyl radical production, measured in PBS by APF assay. PMAO-KI NPs promoted hydroxyl radical generation at different radiation doses. *, p<0.05. FIGS. 2G-2I are line graphs showing ROS production of PMAO-KI NPs under RT. FIG. 2G shows overall ROS generation, measured by detecting methylene blue absorbance decrease. FIG. 2H shows the results of a dihydroethidium (DHE) assay measuring superoxide production in the presence of PMAO-KI NPs compared with PBS control.

FIG. 2I shows the result of a singlet oxygen species generated (SOSG) assay measuring singlet oxygen generation. FIG. 2J shows NIS expression, measured by flow cytometry. In addition to MCF-7, MDA-MB-231 cells, which have a low expression of NIS, were analyzed as a comparison. Incubating with tRA (1 μM) induced NIS expression in MCF-7 cells.

FIG. 3A is a bar graph showing the results of an MTT assay evaluating cytotoxicity of PMAO-KI NPs. Low cytotoxicity was observed when nanoparticle concentration was below 100 μg iodine/mL. FIG. 3B is a bar graph showing iodine uptake, measured by inductively coupled plasma mass spectrometry (ICP-MS). Increased cellular iodine content was observed with PMAO-KI NPs; the uptake was further increased when cells were treated with tRA (1 μM) that promotes NIS expression. *, p<0.05; ***, p<0.001. FIG. 3C is spectra showing cell uptake of PMAO-KI NPs, examined by flow cytometry. Cy5 labeled PMAO-KI NPs were incubated with MCF-7 cells. For comparison, tRA (1 μM) or KClO4 (1 μM) was co-incubated.

FIGS. 4A-4F show the results of testing the radiosensitizing effects of PMAO-KI NPs, in vitro with MCF-7 cells. FIG. 4A is a bar graph showing anit-γH2AX staining results. Cells were incubated with PMAO-KI NPs (25 μg/mL), and irradiated by X-ray (5 Gy). Images were acquired at 24 h. Average foci number per cell was compared. *, p<0.05. FIG. 4B is a bar graph showing lipid peroxidation, evaluated by BODIPY 581/591 assay. *, p<0.05. FIG. 4C is a line graph showing cell viability after the treatment with PMAO-KI NPs+RT w/or w/o tRA. MTT assay was used after 24 h of incubation PMAO-KI NPs at 25 μg·KI/mL with MCF-7 cells at different doses of RT. (*p<0.05). FIG. 4D is a line graph showing dose modifying effects, evaluated on the basis of MTT results. MCF-7 cells were incubated with PMAO-KI NPs (25 μg/mL, in the presence of tRA), and irradiated (5 Gy). Viability drop relative to cells receiving radiation only (without incubation with PMAO-KI NPs) at 24 or 72 hrs was computed and presented. *, p<0.05. A dose dependent response is observed at both time points with 72 hrs showing a greater dose enhancement. FIG. 4E is a line graph showing alternate clonogenic assay representation. Actual survival fractions represented logarithmically. Survival fraction from PMAO-KI NPs+tRA was significantly lower than the PMAO-KI NPs and RT alone (*** p<0.005). FIG. 4F is a line graph showing clonogenic assay results, performed in the presence or absence of 25 μg/mL PMAO-KI NPs. The results were fitted to the liner-quadratic formula, S(D)/S(0)=exp−(aD+bD²), where S is the survival fraction, D is the radiation dose in Gy, and a and b are fitting coefficients. ***, p<0.001. e) Dose modifying factors, based on results from d. D₁₀, dose required for 10% survival. DMR, dose modification ratio based on D₁₀.

FIGS. 5A-5E show the biodistribution of iodine. ¹³¹I-doped PMAO-KI NPs or ¹³¹I-KI was i.t. injected into MCF-7 bearing nude mice. tRA was pre-injected to stimulate NIS expression. Gamma counting was performed at 72 hrs with dissected tumors and normal tissues to examine distribution. FIG. 5A is a histogram comparing the retention of iodine in tumors. Tumor treated with PMAO-KI NPs plus tRA showed the highest activity due to controlled iodine release and uptake through NIS. FIGS. 5B-5D are images of autoradiographs of tumors treated with ¹³¹I-PMAO-KI NPs plus tRA (tRA+KI NPs), ¹³¹I-PMAO-KI NPs (KI NPs), ¹³¹I-KI solution plus tRA (tRA+KI solution). The images were displayed in pseudocolors based on the scale appearing on the right. FIG. 5E is a histogram showing the distribution of iodine in normal tissues.

FIGS. 6A-6C illustrate the results of in vivo therapeutic studies, tested in a MCF7 xenograft model (n=5). PMAO-KI NPs (50 μg iodine/mL in 50 μL PBS) were i.t. injected, followed by 5-Gy beam radiation applied to tumors (PMAO-KI NPs+RT). For comparison, PBS, radiation alone (RT), and PMAO-KI NPs alone (PMAO-KI NPs) were tested. tRA was pre-injected to promote NIS expression. FIG. 6A is a tumor growth curve. Relative to RT alone, PMAO-KI NPs+RT significantly improved tumor suppression. 80% of the treated mice remained tumor-free at the end of the experiment. *, p<0.05. FIG. 6B is an animal survival curve. All animals remained alive in the PMAO-KI NPs+RT group. FIG. 6C is a line graph showing relative body weight changes. No significant body weight changes were observed through the studies.

FIG. 7 is an illustration modeling the mechanisms for PMAO-KI NPs based particle delivery and radiosensitization. I ions are slowly released from PMAO-KI NPs, and internalized by breast cancer cells through the NIS symporter. I promotes ROS generation under radiation, causing extensive DNA and lipid damage as well as reduced cell viability and clonogenicity

### DETAILED DESCRIPTION OF THE INVENTION

### I. Definitions

As used herein, the term “tumor” or “neoplasm” refers to an abnormal mass of tissue containing neoplastic cells. Neoplasms and tumors may be benign, premalignant, or malignant.

As used herein, the term “cancer” or “malignant neoplasm” refers to a cell that displays uncontrolled growth and division, invasion of adjacent tissues, and often metastasizes to other locations of the body.

As used herein, the term “antineoplastic” refers to a composition, such as a drug or biologic, that can inhibit or prevent cancer growth, invasion, and/or metastasis.

As used herein, the term “biocompatible” as used herein refers to one or more materials that are neither themselves toxic to the host (e.g., an animal or human), nor degrade (if the material degrades) at a rate that produces monomeric or oligomeric subunits or other byproducts at toxic concentrations in the host.

As used herein, the term “biodegradable” means that the materials degrade or breaks down into its component subunits, or digestion, e.g., by a biochemical process, of the material into smaller (e.g., non-polymeric) subunits.

As used herein, the term “microparticles” generally refers to a particle having a diameter less than about 1000 microns. The particles can have any shape.

As used herein, the term “nanoparticle” generally refers to a particle having a diameter from about 10 nm up to, but not including, about 1 micron, or from 100 nm to about 1 micron. The particles can have any shape. The particles can be cubic, for example. Other non-limiting shapes which are contemplated can include tetrahedral, bipyramidal, octahedral, icosahedral, and decahedral shapes.

A composition containing microparticles and/or nanoparticles may include particles of a range of particle sizes. In certain embodiments, the particle size distribution may be uniform, e.g., within less than about a 20% standard deviation of the mean volume diameter, and in other embodiments, still more uniform, e.g., within about 10% of the median volume diameter.

As used herein, the phrase “mean particle size” generally refers to the statistical mean particle size (diameter) of the particles in a population of particles. The diameter of an essentially spherical particle may refer to the physical or hydrodynamic diameter. The diameter of a non-spherical particle may refer to the hydrodynamic diameter. As used herein, the diameter of a non-spherical particle may refer to the largest linear distance between two points on the surface of the particle. Mean particle size can be measured using methods known in the art, such as dynamic light scattering or electronic microscopy such as scanning electron microscopy (SEM) or transmission electron microscopy (TEM).

As used herein, the phrases “monodisperse” and “homogeneous size distribution” are used interchangeably and describe a population of nanoparticles or microparticles where all of the particles are the same or nearly the same size. As used herein, a monodisperse distribution refers to particle distributions in which 90% of the distribution lies within 15% of the median particle size, or within 10% of the median particle size, or within 5% of the median particle size.

As used herein “targeting agent” and “targeting moiety” refers to a chemical compound that can direct a particle to a receptor site on a selected cell or tissue type, can serve as an attachment molecule, or serve to couple or attach another molecule. The term “direct,” as relates to chemical compounds, refers to causing a particle to preferentially attach to a selected cell or tissue type. This targeting agent generally binds to its receptor with high affinity and specificity.

As used herein, the phrase “pharmaceutically acceptable” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

As used herein, the phrase “pharmaceutically acceptable salts” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the halides, hydroxides, carbonates, and bicarbonates of ammonia, sodium, lithium, potassium, cesium, calcium, magnesium, aluminum, and zinc. Salts may also be formed with suitable organic bases, including those that are non-toxic and strong enough to form such salts.

As used herein, the term “individual,” “host,” “subject,” and “patient” are used interchangeably to refer to any individual who is the target of administration or treatment. The subject can be a vertebrate, for example, a mammal. Thus, the subject can be a human or veterinary patient.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “therapeutically effective amount” refers to an amount of a therapeutic agent that produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish the symptoms of one or more diseases or disorders, such as reducing tumor size (e.g., tumor volume).

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx.+/−10%; in other forms the values may range in value either above or below the stated value in a range of approx.+/−5%; in other forms the values may range in value either above or below the stated value in a range of approx.+/−2%; in other forms the values may range in value either above or below the stated value in a range of approx.+/−1%. The preceding ranges are intended to be made clear by context, and no further limitation is implied.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

### II. Iodide Particle Compositions

### A. Core Composition

Particles formed from iodide and alkali metal ions, such as potassium, lithium, sodium, rubidium, cesium or strontium, or alkaline earth metal ions, such as magnesium or calcium (referred to as iodide particles), and methods of use thereof are provided.

In preferred embodiments, the particles are potassium iodide (KI) particles, preferably KI nanoparticles. Although the compositions and methods described in detail herein focus primarily upon KI particles, particularly KI nanoparticles, corresponding particle embodiments of iodide particles paired with another alkali metal or alkaline earth metal such as those provided above are also specifically disclosed and can substitute for, or supplement, KI particles in the compositions and methods provided herein.

In some embodiments, a radioisotope such as ¹³¹I, ¹²⁵I, ¹²⁴I, or ¹²³I is incorporated into iodide particles, producing “hot” particles that can serve as a radiopharmaceutical or a brachytherapy agent for cancer treatment, or an imaging agent.

Thus, these NPs can be injected into tumors, e.g., a breast tumor, to enable sustained iodide release and cellular uptake.

The disclosed particles are typically nanoscale in size, for example, having a diameter of 10 nm up to, but not including, about 1 micron. However, it will be appreciated that in some embodiments, and for some uses, the particles can be smaller or larger (e.g., microparticles, etc.). Although many of the compositions disclosed herein are referred to as nanoparticle compositions, it will be appreciated that in some embodiments and for some uses the carrier can be somewhat larger than nanoparticles. For example, carrier compositions can also include particles having a diameter of between about 1 micron to about 1000 microns. Such compositions can be referred to as microparticle compositions.

Nanoparticles are often utilized for intratissue applications and penetration of cells. Thus, in some embodiments, the particles are nanoparticles that have any diameter from 10 nm up to about 1,000 nm, or any subrange or specific integer therebetween. For example, the nanoparticles can have a diameter from 10 nm to 900 nm, from 10 nm to 800 nm, from 10 nm to 700 nm, from 10 nm to 600 nm, from 10 nm to 500 nm, from 20 nm from 500 nm, from 30 nm to 500 nm, from 40 nm to 500 nm, from 50 nm to 500 nm, from 50 nm to 400 nm, from 50 nm to 350 nm, from 50 nm to 300 nm, or from 50 nm to 200 nm, from 10 nm to 100 nm. For example, in some embodiments, the particles are about 15 nm, 25 nm, 60 nm, 100 nm, or any other integer value or range of values between 1 nm and 1000 nm inclusive. In some embodiments the nanoparticles can have a diameter less than 400 nm, less than 300 nm, or less than 200 nm. For example, the nanoparticle can have a diameter from between 50 nm and 300 nm.

The disclosed sizes can be the particle size with or without a polymer coating. Thus, in some embodiments, the sizes are the average diameters of the iodide particle core.

In one example, the average diameters of the core of iodide nanoparticles are between about 15 nm and about 800 nm, or between about 20 nm and about 500 nm, or between about 50 nm and about 350 nm, or any subrange or specific integer there between. In some embodiments, the average diameters of the nanoparticles are about 50 nm to about 125 nm, or about 80 nm.

Preferably the particles of a size that can be internalized by cancer cell, preferably through the NIS.

Particles size can be measure or determined by, for example, dynamic light scattering, electronic microscopy such as scanning electron microscopy (SEM), and transmission electron microscopy (TEM).

In some embodiments the iodide particles in a particle composition are monodispersed. In some embodiments, the iodide particles in a particle composition are of various sizes (i.e., polydispersed).

### B. Coating

KI nanoparticles synthesized as described below can be hydrophobic because of e.g., an oleylamine coating. Furthermore, although iodine has shown promise in enhancing radiotherapy, conventional iodine compounds show fast clearance and low retention inside cancer cells, limiting their application as a radiosensitizer. For iodide salts such as KI, systemic injection would result in overwhelming iodine accumulation in the thyroid. Another challenge is that iodides have a high water mobility and would be quickly cleared from the injection site. Nanoparticles will reduce thyroid uptake and increase tumor uptake of iodine. To further enhance the nanoparticles, a coating can be added to make them.

Thus, the particles optionally, but preferably, include a coating. Also referred to herein a layer, or external layer, the coating/layer is typically over the iodide core of the particles. In some embodiments, the coating enhances the particles' compatibility with aqueous solutions. Additionally or alternatively, the coating can be added to extend the half-lives of the nanoparticle in aqueous environments and/or improve nanoparticle uptake by cells. The Examples below show that a polymer coating over KI core particles is not instantly dissolved in aqueous solutions but degrades slowly, allowing for controlled release of iodide (I⁻). I⁻ is transported into cells via the sodium iodide symporter (NIS), which is upregulated in breast cancer cells.

### 1. Composition of the Coating

The coating can be composed of, for example, polar or non-polar polymers and co-polymers, peptides, proteins, lipids, silica, metal oxides, or combinations thereof. In some embodiments, the coating is composed of conjugates or fusions of two or more of the foregoing alone or in further combination with one or more active agents and/or targeting moieties.

In preferred embodiments, the coating is formed of poly(maleic anhydride alt-1-octadecene) (PMAO). In the experiments below, KI NPs were synthesized and coated with PMAO. PMAO is non-polar polymer that is biodegradable, biocompatible, and readily coupled with other molecules (Karandikar, et al., Eds, 263-293 (2017)). Results show that the polymer coating extended the half-lives of the electrolyte NPs in aqueous solutions. The controlled release coating addresses the short circulation half-lives and low tumor uptake seen with other iodine-containing molecules such as Iomeprol. In the clinic, breast cancer patients after mastectomy or lumpectomy often receive multi-session fractionated RT to prevent recurrence. It is envisioned that KI NPs can be injected e.g., into the tumor bed for sustained iodide release and radiosensitization. The PMAO-KI formulation exemplified in the experiments below has t_{1/2} of ˜24 h. However, is possible to increase the coating thickness and/or degree of crosslinking to extend iodide release so that one injection can benefit multiple RT sessions.

For example, in some embodiments, the thickness of the coating ranges from 1 nm to 200 nm, or 10 nm to 100 nm, or 25 nm to 75 nm inclusive, or any subrange or specific integer therebetween, such as 50 nm. While PMAO is a preferred coating, other coatings are also contemplated, and examples are discussed below.

a. Polymers

In some embodiments, the layer or coating around the particles is formed of one or more polymers. The polymer can be polar, non-polar, or amphiphilic, and can be a single polymer or a copolymer. Polymer refers to a molecular structure including one or more repeat units (monomers), connected by covalent bonds. A biocompatible polymer refers to a polymer that does not typically induce an adverse response when inserted or injected into a living subject. A copolymer refers to a polymer formed of two or more different monomers. The different units may be arranged in a random order, in an alternating order, or as a “block” copolymer, i.e., including one or more regions each including a first repeat unit (e.g., a first monomer or block of monomers), and one or more regions each including a second repeat unit (e.g., a second block), etc. Block copolymers may have two (a diblock copolymer), three (a triblock copolymer), or more numbers of distinct blocks.

The term “amphiphilic” refers to a molecule that has both a polar portion and a non-polar portion. In some embodiments, the polar portion (e.g., a hydrophilic portion such as a hydrophilic polymer) is soluble in water, while the non-polar portion (e.g., a hydrophobic portion such as a hydrophobic polymer) is insoluble in water. The polar portion may have either a formal positive charge, or a formal negative charge. Alternatively, the polar portion may have both a formal positive and a negative charge, and be a zwitterion or inner salt.

The hydrophilic portion of the amphiphilic material can form a corona around the particle that increases the particle's solubility in aqueous solution. In a particular embodiment, the amphiphilic material is a hydrophobic, biodegradable polymer terminated with a hydrophilic block.

The hydrophilic portion and hydrophobic portion can be biocompatible hydrophilic and hydrophobic polymers respectively. Exemplary biocompatible polymers include, but are not limited to, polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polylactides, polyglycolides, polysiloxanes, polyurethanes and copolymers thereof, celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt; polyacrylic acid polymers such as polymers of acrylic and methacrylic esters such as poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyalkylenes such as polyethylene, polypropylene poly(ethylene glycol), poly(ethylene oxide), and poly(ethylene terephthalate), poly(vinyl alcohols), poly(vinyl acetate), poly vinyl chloride polystyrene and polyvinylpryrrolidone, derivatives thereof, linear and branched copolymers and block copolymers thereof, and blends thereof.

Other exemplary biodegradable polymers include, but are not limited to, polyesters, polydopamine, poly(ortho esters), poly(ethylene imines), poly(caprolactones), poly(hydroxybutyrates), poly(hydroxyvalerates), polyanhydrides, poly(acrylic acids), polyglycolides, poly(urethanes), polycarbonates, polyphosphate esters, polyphosphazenes, derivatives thereof, linear and branched copolymers and block copolymers thereof, and blends thereof. In particularly preferred embodiments the co-polymer include one or more biodegradable hydrophobic polyesters such as poly(lactic acid), poly(glycolic acid), and poly(lactic-co-glycolic acid), and/or these polymers conjugated to polyalkylene oxides such as polyethylene glycol or block copolymers such as the polypropylene oxide-polyethylene oxide PLURONICS®.

The molecular weight of the biodegradable oligomeric or polymeric segment or polymer can be varied to tailor the properties of the polymer. In some embodiments, the hydrophilic polymers or segment(s) or block(s) include, but are not limited to, homo polymers or copolymers of polyalkene glycols, such as poly(ethylene glycol), poly(propylene glycol), poly(butylene glycol), and acrylates and acrylamides, such as hydroxyethyl methacrylate and hydroxypropyl-methacrylamide.

The hydrophobic portion of amphiphilic materials can provide a non-polar polymer matrix coating for loading non-polar drugs.

b. Lipids

The coating can be, or include, one or more lipids. Lipids and other components useful in preparing the disclosed nanoparticle compositions having a lipid-based coating are known in the art. Suitable neutral, cationic and anionic lipids include, but are not limited to, sterols and lipids such as cholesterol, phospholipids, lysolipids, lysophospholipids, and sphingolipids. Neutral and anionic lipids include, but are not limited to, phosphatidylcholine (PC) (such as egg PC, soy PC), including, but limited to, 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; phosphoethanolamines such as 1,2-distearoyl-sn-glycero-3-phosphoethanolamine (DSPE), 1,2-diacyl-sn-glycero-3-phosphoethanolamine, including, but not limited to, 1,2-dioleylphosphoethanolamine (DOPE), 1,2-dihexadecylphosphoethanolamine (DHPE); and phophatidylcholines such as 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids.

The lipid can be a sphingomyelin metabolites such as, without limitation, ceramide, sphingosine, or sphingosine 1-phosphate.

Exemplary catonic lipids include, but are not limited to, N-[1-(2,3-dioleoyloxy) propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy) propyl]-N,N-dimethyl amine (DODAP), 1,2-diacyloxy-3-dimethylammonium propanes, N-[1-(2,3-dioleyloxy) propyl]-N,N,N-trimethylammonium chloride (DOTMA), 1,2-dialkyloxy-3-dimethylammonium propanes, dioctadecylamidoglycylspermine (DOGS), 3-[N-(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl) diethanolamine chloride, 1,3-dioleoyloxy-2-(6-carboxy-spermyl)-propylamide (DOSPER), and N, N, N′, N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9 (Z)-octadecenoyloxy)ethyl]-2-(8 (Z)-heptadecenyl-3-(2-hydroxyethyl) imidazolinium chloride (DOTIM), and 1-[2-(hexadecanoyloxy)ethyl]-2-pentadecyl-3-(2-hydroxyethyl) imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

The lipids can be formed from a combination of more than one lipid, for example, a charged lipid may be combined with a lipid that is non-ionic or uncharged at physiological pH. Non-ionic lipids include, but are not limited to, cholesterol and DOPE (1,2-dioleolylglyceryl phosphatidylethanolamine).

A sterol component may be included to confer a physicochemical and biological behavior. Such a sterol component may be selected from cholesterol or its derivative e.g., ergosterol or cholesterolhemisuccinate.

The coating can include a single type of lipid, or a combination of two or more lipids.

c. Polyethers and Polyquaterniums

The coating can be, or include, a polyether. Exemplary polyethers include, but are not limited to, oligomers and polymers of ethylene oxide. In preferred embodiments, the polyether is a Polyethylene glycol (PEG). PEGs are prepared by polymerization of ethylene oxide and are commercially available over a wide range of molecular weights from 300 g/mol to 10,000,000 g/mol, and can have branched, star, or comb geometries. The numbers that are often included in the names of PEGs indicate their average molecular weights (e.g. a PEG with n=9 would have an average molecular weight of approximately 400 daltons, and would be labeled PEG 400.) Most PEGs include molecules with a distribution of molecular weights (i.e., they are polydisperse). The size distribution can be characterized statistically by its weight average molecular weight (Mw) and its number average molecular weight (Mn), the ratio of which is called the polydispersity index (Mw/Mn). Mw and Mn can be measured by mass spectrometry. In some embodiment the PEG is an amino (polyethylene glycol) (also referred to as a PEG amine).

In some embodiments, the PEG or PEG amine is up about 25,000, or more. In some embodiments, the PEG or PEG amine is about PEG 350 to about PEG 25,000, or about PEG 350 to about PEG 20,000. In some embodiments, the PEG or PEG amine is about PEG 350 to about PEG 5000, or between about PEG 750 and about PEG 5000, or between about PEG 1000 and PEG 3000. In a particular embodiment, the PEG is PEG 2000.

In particular embodiments, the coating is a polyether-lipid (e.g., phospholipid) conjugate coating. In some embodiments, the polyether-phospholipid conjugate is DSPE-PEG2000 amine. See, for example, the experiments below which describe coating DSPE-PEG2000 amine, onto the nanoparticle surface.

In some embodiments, the coating includes or is formed of one or more polyquaterniums. Polyquaternium is the International Nomenclature for Cosmetic Ingredients designation for several polycationic polymers that are used in the personal care industry. Polyquaternium is a neologism used to emphasize the presence of quaternary ammonium centers in the polymer. INCI has approved at least 40 different polymers under the polyquaternium designation. Different polymers are distinguished by the numerical value that follows the word “polyquaternium”, and include, e.g., polyquaternium-1 through polyquaternium-20, polyquaternium-22, polyquaternium-24, polyquaternium-27 through polyquaternium-37, polyquaternium-39, and polyquaternium-42 through polyquaternium-47. In particular embodiments, the polyquaternium is polyquaternium-7, -10, or -30.

### 2. Targeting Agents

It is also possible to inject KI NPs systemically and rely on either passive or active targeting of NPs for tumor accumulation (Golombek, et al., Adv Drug Deliv Rev, 130, 17-38 (2018)). Thus, in some embodiments, the particles include a targeting agent, most typically conjugated to one or more components of the coating.

The particles can contain a targeting moiety, as referred to herein as a targeting agent. The targeting moiety can specifically recognize and bind to a target molecule specific for a cell type, a tissue type, or an organ. The target molecule can be a cell surface polypeptide, lipid, or glycolipid. The target molecule can be a receptor that is selectively expressed on a specific cell surface, a tissue or an organ. Cell specific markers can be for specific types of cells including, but not limited to stem cells, skin cells, blood cells, immune cells, muscle cells, nerve cells, cancer cells, virally infected cells, bacterial cells, fungal cells, and organ specific cells. The cell markers can be specific for endothelial, ectodermal, or mesenchymal cells. Representative cell specific markers include, but are not limited to cancer specific markers.

The targeting moiety can be a peptide. The targeting peptides can be covalently associated with the polymer and the covalent association can be mediated by a linker.

The particles can contain a targeting agent, for example an antigen targeting agent. In some embodiments, the targeting agents bind to antigens, ligands or receptors that are specific to tumor cells or tumor-associated neovasculature, or are upregulated in tumor cells or tumor-associated neovasculature compared to normal tissue.

In some embodiment the particles contain a domain that specifically binds to an antigen that is expressed by tumor cells. The antigen expressed by the tumor may be specific to the tumor, or may be expressed at a higher level on the tumor cells as compared to non-tumor cells. Antigenic markers such as serologically defined markers known as tumor associated antigens, which are either uniquely expressed by cancer cells or are present at markedly higher levels (e.g., elevated in a statistically significant manner) in subjects having a malignant condition relative to appropriate controls, are contemplated for use in certain embodiments.

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). Other tumor-associated antigens include molecules that may be directly involved in transformation events, or molecules that may not be directly involved in oncogenic transformation events but are expressed by tumor cells (e.g., carcinoembryonic antigen, CA-125, melonoma associated antigens, etc.) (see, e.g., U.S. Pat. No. 6,699,475; Jager, et al., Int. J. Cancer, 106:817-20 (2003); Kennedy, et al., Int. Rev. Immunol., 22:141-72 (2003); Scanlan, et al. Cancer Immun., 4:1 (2004)).

Genes that encode cellular tumor associated antigens include cellular oncogenes and proto-oncogenes that are aberrantly expressed. In general, cellular oncogenes encode products that are directly relevant to the transformation of the cell. An example is the tumorigenic neu gene that encodes a cell surface molecule involved in oncogenic transformation. Other examples include the ras, kit, and trk genes. The products of proto-oncogenes (the normal genes which are mutated to form oncogenes) may be aberrantly expressed (e.g., overexpressed), and this aberrant expression can be related to cellular transformation. Thus, the product encoded by proto-oncogenes can be targeted. Some oncogenes encode growth factor receptor molecules or growth factor receptor-like molecules that are expressed on the tumor cell surface. An example is the cell surface receptor encoded by the c-erbB gene. Other tumor-associated antigens may or may not be directly involved in malignant transformation. These antigens, however, are expressed by certain tumor cells and may therefore provide effective targets. Some examples are carcinoembryonic antigen (CEA), CA 125 (associated with ovarian carcinoma), and melanoma specific antigens.

In ovarian and other carcinomas, for example, tumor associated antigens are detectable in samples of readily obtained biological fluids such as serum or mucosal secretions. One such marker is CA125, a carcinoma associated antigen that is also shed into the bloodstream, where it is detectable in serum (e.g., Bast, et al., N. Eng. J. Med., 309:883 (1983); Lloyd, et al., Int. J. Canc., 71:842 (1997). CA125 levels in serum and other biological fluids have been measured along with levels of other markers, for example, carcinoembryonic antigen (CEA), squamous cell carcinoma antigen (SCC), tissue polypeptide specific antigen (TPS), sialyl TN mucin (STN), and placental alkaline phosphatase (PLAP), in efforts to provide diagnostic and/or prognostic profiles of ovarian and other carcinomas (e.g., Sarandakou, et al., Acta Oncol., 36:755 (1997); Sarandakou, et al., Eur. J. Gynaecol. Oncol., 19:73 (1998); Meier, et al., Anticancer Res., 17(4B): 2945 (1997); Kudoh, et al., Gynecol. Obstet. Invest., 47:52 (1999)). Elevated serum CA125 may also accompany neuroblastoma (e.g., Hirokawa, et al., Surg. Today, 28:349 (1998), while elevated CEA and SCC, among others, may accompany colorectal cancer (Gebauer, et al., Anticancer Res., 17(4B):2939 (1997)).

The tumor associated antigen, mesothelin, defined by reactivity with monoclonal antibody K−1, is present on a majority of squamous cell carcinomas including epithelial ovarian, cervical, and esophageal tumors, and on mesotheliomas (Chang, et al., Cancer Res., 52:181 (1992); Chang, et al., Int. J. Cancer, 50:373 (1992); Chang, et al., Int. J. Cancer, 51:548 (1992); Chang, et al., Proc. Natl. Acad. Sci. USA, 93:136 (1996); Chowdhury, et al., Proc. Natl. Acad. Sci. USA, 95:669 (1998)). Using MAb K-1, mesothelin is detectable only as a cell-associated tumor marker and has not been found in soluble form in serum from ovarian cancer patients, or in medium conditioned by OVCAR-3 cells (Chang, et al., Int. J. Cancer, 50:373 (1992)). Structurally related human mesothelin polypeptides, however, also include tumor-associated antigen polypeptides such as the distinct mesothelin related antigen (MRA) polypeptide, which is detectable as a naturally occurring soluble antigen in biological fluids from patients having malignancies (see WO 00/50900).

A tumor antigen may include or be a cell surface molecule. Tumor antigens of known structure and having a known or described function, include the following cell surface receptors: HER1 (GenBank Accession No. U48722), HER2 (Yoshino, et al., J. Immunol., 152:2393 (1994); Disis, et al., Canc. Res., 54:16 (1994); GenBank Acc. Nos. X03363 and M17730), HER3 (GenBank Acc. Nos. U29339 and M34309), HER4 (Plowman, et al., Nature, 366:473 (1993); GenBank Acc. Nos. L07868 and T64105), epidermal growth factor receptor (EGFR) (GenBank Acc. Nos. U48722, and KO3193), vascular endothelial cell growth factor (GenBank No. M32977), vascular endothelial cell growth factor receptor (GenBank Acc. Nos. AF022375, 1680143, U48801 and X62568), insulin-like growth factor-I (GenBank Acc. Nos. X00173, X56774, X56773, X06043, European Patent No. GB 2241703), insulin-like growth factor-II (GenBank Acc. Nos. X03562, X00910, M17863 and M17862), transferrin receptor (Trowbridge and Omary, Proc. Nat. Acad. USA, 78:3039 (1981); GenBank Acc. Nos. X01060 and M11507), estrogen receptor (GenBank Acc. Nos. M38651, X03635, X99101, U47678 and M12674), progesterone receptor (GenBank Acc. Nos. X51730, X69068 and M15716), follicle stimulating hormone receptor (FSH-R) (GenBank Acc. Nos. Z34260 and M65085), retinoic acid receptor (GenBank Acc. Nos. L12060, M60909, X77664, X57280, X07282 and X06538), MUC-1 (Barnes, et al., Proc. Nat. Acad. Sci. USA, 86:7159 (1989); GenBank Acc. Nos. M65132 and M64928) NY-ESO-1 (GenBank Acc. Nos. AJ003149 and U87459), NA 17-A (PCT Publication No. WO 96/40039), Melan-A/MART-1 (Kawakami, et al., Proc. Nat. Acad. Sci. USA, 91:3515 (1994); GenBank Acc. Nos. U06654 and U06452), tyrosinase (Topalian, et al., Proc. Nat. Acad. Sci. USA, 91:9461 (1994); GenBank Acc. No. M26729; Weber, et al., J. Clin. Invest, 102:1258 (1998)), Gp-100 (Kawakami, et al., Proc. Nat. Acad. Sci. USA, 91:3515 (1994); GenBank Acc. No. S73003, Adema, et al., J. Biol. Chem., 269:20126 (1994)), MAGE (van den Bruggen, et al., Science, 254:1643 (1991)); GenBank Acc. Nos. U93163, AF064589, U66083, D32077, D32076, D32075, U10694, U10693, U10691, U10690, U10689, U10688, U10687, U10686, U10685, L18877, U10340, U10339, L18920, U03735 and M77481), BAGE (GenBank Acc. No. U19180; U.S. Pat. Nos. 5,683,886 and 5,571,711), GAGE (GenBank Acc. Nos. AF055475, AF055474, AF055473, U19147, U19146, U19145, U19144, U19143 and U19142), any of the CTA class of receptors including in particular HOM-MEL-40 antigen encoded by the SSX2 gene (GenBank Acc. Nos. X86175, U90842, U90841 and X86174), carcinoembryonic antigen (CEA, Gold and Freedman, J. Exp. Med., 121:439 (1985); GenBank Acc. Nos. M59710, M59255 and M29540), and PyLT (GenBank Acc. Nos. J02289 and J02038); p97 (melanotransferrin) (Brown, et al., J. Immunol., 127:539-46 (1981); Rose, et al., Proc. Natl. Acad. Sci. USA, 83:1261-61 (1986)).

Additional tumor associated antigens include prostate surface antigen (PSA) (U.S. Pat. Nos. 6,677,157; 6,673,545); B-human chorionic gonadotropin β-HCG) (McManus, et al., Cancer Res., 36:3476-81 (1976); Yoshimura, et al., Cancer, 73:2745-52 (1994); Yamaguchi, et al., Br. J. Cancer, 60:382-84 (1989): Alfthan, et al., Cancer Res., 52:4628-33 (1992)); glycosyltransferase β-1,4-N-acetylgalactosaminyltransferases (GalNAc) (Hoon, et al., Int. J. Cancer, 43:857-62 (1989); Ando, et al., Int. J. Cancer, 40:12-17 (1987); Tsuchida, et al., J. Natl. Cancer, 78:45-54 (1987); Tsuchida, et al., J. Natl. Cancer, 78:55-60 (1987)); NUC18 (Lehmann, et al., Proc. Natl. Acad. Sci. USA, 86:9891-95 (1989); Lehmann, et al., Cancer Res., 47:841-45 (1987)); melanoma antigen gp75 (Vijayasardahi, et al., J. Exp. Med., 171:1375-80 (1990); GenBank Accession No. X51455); human cytokeratin 8; high molecular weight melanoma antigen (Natali, et al., Cancer, 59:55-63 (1987); keratin 19 (Datta, et al., J. Clin. Oncol., 12:475-82 (1994)).

Tumor antigens of interest include antigens regarded in the art as “cancer/testis” (CT) antigens that are immunogenic in subjects having a malignant condition (Scanlan, et al., Cancer Immun., 4:1 (2004)). CT antigens include at least 19 different families of antigens that contain one or more members and that are capable of inducing an immune response, including but not limited to MAGEA (CT1); BAGE (CT2); MAGEB (CT3); GAGE (CT4); SSX (CT5); NY-ESO-1 (CT6); MAGEC (CT7); SYCP1 (C8); SPANXB1 (CT11.2); NA88 (CT18); CTAGE (CT21); SPA17 (CT22); OY-TES-1 (CT23); CAGE (CT26); HOM-TES-85 (CT28); HCA661 (CT30); NY-SAR-35 (CT38); FATE (CT43); and TPTE (CT44).

Additional tumor antigens that can be targeted, including a tumor-associated or tumor-specific antigen, include, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARa fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15 (58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, a-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS. Other tumor-associated and tumor-specific antigens are known to those of skill in the art and are suitable for targeting by the disclosed fusion proteins.

In other embodiments, the antigen is one that is expressed by neovasculature associated with a tumor. The antigen may be specific to tumor neovasculature or may be expressed at a higher level in tumor neovasculature when compared to normal vasculature. Exemplary antigens that are over-expressed by tumor-associated neovasculature as compared to normal vasculature include, but are not limited to, VEGF/KDR, Tie2, vascular cell adhesion molecule (VCAM), endoglin and α₅β₃ integrin/vitronectin. Other antigens that are over-expressed by tumor-associated neovasculature as compared to normal vasculature are known to those of skill in the art and are suitable for targeting by the disclosed fusion proteins.

In other embodiments, the fusion proteins contain a domain that specifically binds to a chemokine or a chemokine receptor. Chemokines are soluble, small molecular weight (8-14 kDa) proteins that bind to their cognate G-protein coupled receptors (GPCRs) to elicit a cellular response, usually directional migration or chemotaxis. Tumor cells secrete and respond to chemokines, which facilitate growth that is achieved by increased endothelial cell recruitment and angiogenesis, subversion of immunological surveillance and maneuvering of the tumoral leukocyte profile to skew it such that the chemokine release enables the tumor growth and metastasis to distant sites. Thus, chemokines are vital for tumor progression.

Based on the positioning of the conserved two N-terminal cysteine residues of the chemokines, they are classified into four groups namely CXC, CC, CX3C and C chemokines. The CXC chemokines can be further classified into ELR+ and ELR− chemokines based on the presence or absence of the motif ‘glu-leu-arg (ELR motif)’ preceding the CXC sequence. The CXC chemokines bind to and activate their cognate chemokine receptors on neutrophils, lymphocytes, endothelial and epithelial cells. The CC chemokines act on several subsets of dendritic cells, lymphocytes, macrophages, eosinophils, natural killer cells but do not stimulate neutrophils as they lack CC chemokine receptors except murine neutrophils. There are approximately 50 chemokines and only 20 chemokine receptors, thus there is considerable redundancy in this system of ligand/receptor interaction.

Chemokines elaborated from the tumor and the stromal cells bind to the chemokine receptors present on the tumor and the stromal cells. The autocrine loop of the tumor cells and the paracrine stimulatory loop between the tumor and the stromal cells facilitate the progression of the tumor. Notably, CXCR2, CXCR4, CCR2 and CCR7 play major roles in tumorigenesis and metastasis. CXCR2 plays a vital role in angiogenesis and CCR2 plays a role in the recruitment of macrophages into the tumor microenvironment. CCR7 is involved in metastasis of the tumor cells into the sentinel lymph nodes as the lymph nodes have the ligand for CCR7, CCL21. CXCR4 is mainly involved in the metastatic spread of a wide variety of tumors.

### Molecular Classes of Targeting Domains

### Ligands and Receptors

In one embodiment, tumor or tumor-associated neovasculature targeting agents are ligands that bind to cell surface antigens or receptors that are specifically expressed on tumor cells or tumor-associated neovasculature or are overexpressed on tumor cells or tumor-associated neovasculature as compared to normal tissue. Tumors also secrete a large number of ligands into the tumor microenvironment that affect tumor growth and development. Receptors that bind to ligands secreted by tumors, including, but not limited to growth factors, cytokines and chemokines, including the chemokines provided above, are suitable for use in the disclosed fusion proteins. Ligands secreted by tumors can be targeted using soluble fragments of receptors that bind to the secreted ligands. Soluble receptor fragments are fragments polypeptides that may be shed, secreted or otherwise extracted from the producing cells and include the entire extracellular domain, or fragments thereof.

### Antibodies

In another embodiment, tumor or tumor-associated neovasculature targeting domains are antibodies, such as single polypeptide antibodies that bind to cell surface antigens or receptors that are specifically expressed on tumor cells or tumor-associated neovasculature or are overexpressed on tumor cells or tumor-associated neovasculature as compared to normal tissue. The term “antibody” refers to natural or synthetic antibodies that selectively bind a target antigen. The term includes polyclonal and monoclonal antibodies. The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody or a fragment, variant, or fusion protein thereof. The antibody can be in monomeric or polymeric form.

In addition to intact immunoglobulin molecules, also included in the term “antibodies” are fragments or polymers or fusions of those immunoglobulin molecules, and human or humanized versions of immunoglobulin molecules that selectively bind the target antigen. Exemplary fragments and fusions include, but are not limited to, single chain antibodies, single chain variable fragments (scFv), di-scFv, tri-scFv, diabody, triabody, teratbody, disulfide-linked Fvs (sdFv), Fab′, F(ab′)₂, Fv, and single domain antibody fragments (sdAb).

In some embodiments, the targeting moeity includes two or more scFv. For example, the targeting moiety can be a scFv or a di-scFv.

### Aptamers

In some embodiments, the targeting agent is an aptamer. Aptamers are oligonucleotide or peptide sequences with the capacity to recognize virtually any class of target molecules with high affinity and specificity. Aptamers bind to targets such as small organics, peptides, proteins, cells, and tissues. Unlike antibodies, some aptamers exhibit stereoselectivity. The aptamers can be designed to bind to specific targets expressed on cells, tissues or organs.

### 3. Active Agents

The disclosed iodide particles can have a molecular and even therapeutic effect without any additional active agent, and thus in some embodiments, the iodide particles alone are the active material and the particles do not include (i.e., are free from) an additional active agent. Alternatively, the particle can optionally include one or more active agent. For example, in some embodiments, the outer layer or coating is, or includes an active agent. In some embodiments, the active agent or agents are conjugated to a component of the hydrophilic layer or otherwise attached to the surface of the layer, or incorporated, loaded or encapsulated into the layer itself. In some such embodiments, the iodide core of the particles remains free of additional active agents.

In an exemplary embodiment, the coating includes lipids and the active agent or agent(s) are loaded or otherwise incorporated into or beneath the lipid layer, for example by adding the active agent to the reaction mixture when the lipid components are added to the surface of the iodide particles.

The active agent or agents can be, for example, nucleic acids, proteins, and/or small molecules. Exemplary active agents include, for example, tumor antigens, CD4+ T-cell epitopes, cytokines, chemotherapeutic agents, radionuclides, small molecule signal transduction inhibitors, photothermal antennas, immunologic danger signaling molecules, other immunotherapeutics, enzymes, antibiotics, antivirals, anti-parasites (helminths, protozoans), growth factors, growth inhibitors, hormones, hormone antagonists, antibodies and bioactive fragments thereof (including humanized, single chain, and chimeric antibodies), antigen and vaccine formulations (including adjuvants), peptide drugs, anti-inflammatoires, immunomodulators (including ligands that bind to Toll-Like Receptors (including but not limited to CpG oligonucleotides) to activate the innate immune system, molecules that mobilize and optimize the adaptive immune system, molecules that activate or up-regulate the action of cytotoxic T lymphocytes, natural killer cells and helper T-cells, and molecules that deactivate or down-regulate suppressor or regulatory T-cells), agents that promote uptake of the delivery vehicle into cells (including dendritic cells and other antigen-presenting cells), nutraceuticals such as vitamins, and oligonucleotide drugs (including DNA, RNAs, antisense, aptamers, small interfering RNAs, ribozymes, external guide sequences for ribonuclease P, and triplex forming agents).

### C. Methods of Manufacture

### 1. Methods of Making Iodide Core Particles

The disclosed particles are preferably formed of potassium and iodide, though others such as those mentioned above are also specifically contemplated.

For KI nanoparticle (KI NP) synthesis, the particles are typically formed in organic solvents using appropriate potassium and iodide precursors, with or without a radioisotope such as ¹³¹I, ¹²⁵I, ¹²⁴I, or ¹²³I. For instance, potassium oleate reacts with I₂ in octadecene in the presence of oleylamine at room temperature. It is believed that oleate and oleylamine function in collaboration to provide reverse micelle structures, within which KI nucleates and forms particles (Sun, et al., Science 2000, 287 (5460), 1989-1992 (2000)). Oleylamine also functions as a mild reducing agent to convert I₂ to I⁻ (Xu, et al., Chemistry of Materials, 21 (9), 1778-1780 (2009)). Typical reactions yielded monodisperse KI NPs. By adjusting precursor amounts of potassium oleate and I₂, KI NPs in the range of at least 20-500 nm can be produced.

Examples of other potassium precursors include, but are not limited to, potassium chloride (KCl), potassium bromide (KBr), potassium carbonate (K2CO3), potassium nitrate (KNO3), potassium hydroxide (KOH), potassium acetate (CH3CO2K), other potassium fatty salts of fatty acids, and alkoxides of potassium, sulfonates of potassium, and organophosphates of potassium.

Example of other iodide precursors include, but are not limited to, povidone-iodine, 4-Iodopyrazole, and pentacosafluoro-1-iodododecane.

Example of other solvents include, but are not limited to, hexane, ethers such as benzyl ether, haloalkanes, ester, alcohol, aliphatic hydrocarbons, aromatic hydrocarbons, acetone, acetonitrile, aniline, etc.

For example, in the experiments disclosed below, KI particles were prepared by adding potassium oleate to a round bottom flask with 1-Octadecene and mixing at 290° C. until oleate was fully solubilized. Once the reaction was cooled oleylamine was added and mixed, followed by Iodine and the reaction was sealed and mixed. NPs were centrifuged at 12,096×g for 10 mins. The particles are then washed three times in EtOH and placed in an oven until dry.

As exemplified in more below, for preparation of radioisotopic particles, radioisotopic I-NaI (e.g., ¹³¹I-NaI, ¹²⁵I-NaI, ¹²⁴I-NaI, ¹²³I-NaI, etc.) can be suspended in an organic solvent and mixed with acetyl iodide, before combining the mixture with a solution formed of potassium precursor such as potassium acetate and EtOH, hexane, and oleyamine. White precipitates can be selected from the brown supernatant and dried.

### 2. Methods Coating Core Particles

In an exemplary coating method, previously synthesized KI NPs in solvent (e.g., chloroform) are added to the mixture and sonicated into suspension, after which poly(maleic anhydride alt-1-octadecene) (PMAO) in solvent and PEG-bis-amine can be added and the solution mixed. After solvent removal, bis-hexamethyl triamine in solvent can be added to the flask and sonicated until the film was resuspended. After drying, buffer (e.g., 50 mM borate) can be added to cover the dried film and sonicated until film was fully in solution, after which the solution can be filtered to remove excessive aggregates then centrifuged. Supernatant can be discarded and precipitate can be dried (e.g., below 120° C. to avoid polymer softening). The dried and sealed PMAO-KI NPs can be stored for at least 6 months with no discernable loss in quality.

Preferably, the coated nanoparticles described have extended lifetimes of e.g., between about 1 and 48 hours, about 1 and 24 hours, about 1 and 12 hours. In some instances, the coated nanoparticles described have extended lifetimes of at least about 0.5, 1, 2, 3, 4, 5, 6,7,8,9, 10, 11, 12, 13, 14, 15, 16, 17, 18, 19, 20, 21, 22, 23, 24, 30, 35, 40, 48 hours, or longer.

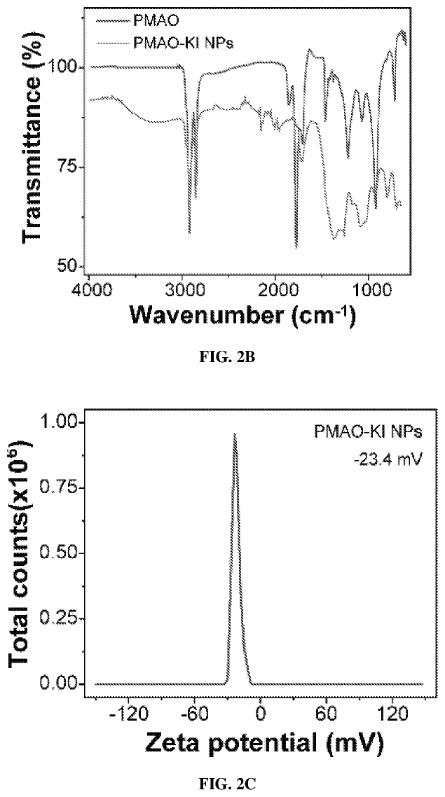

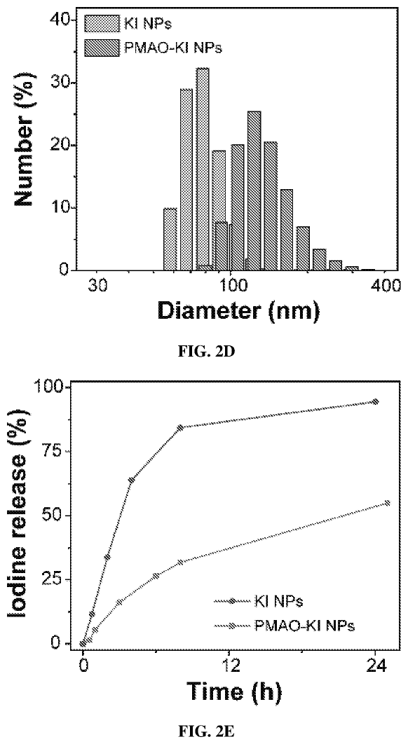

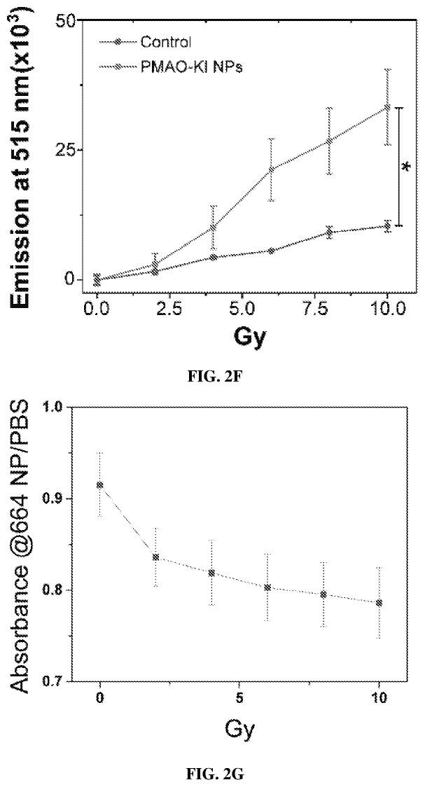

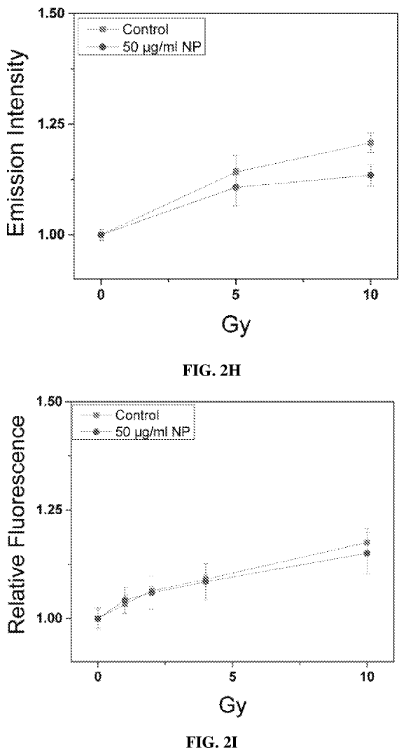

The results below illustrate that the time it took for half of the iodine to be released from PMAO-KI NPs, or t_{1/2}, is negatively correlated with the coating thickness. The formulation tested below included a coating thickness of ˜50 nm and a t_{1/2} of ˜24 hours (FIG. 2e).

The particle coatings described can impart a surface charge on the coated iodide particles. In some instances, the coated particles have a zeta potential of between about −60 mV and about +60 mV, −50 mV and about +50 mV, −40 mV and about +40 mV, −30 mV and about +30 mV, between about −20 mV and about +20 mV, or between about −10 mV and about +10 mV, or any subrange or specific integer therebetween.

In some embodiments, the particle has a negative charge, e.g., −60 mV and about 0 mV, −50 mV and about 0 mV, −40 mV and about 0 mV, or −30 mV and about 0 mV, or any subrange or specific integer therebetween. In some instances, the zeta potential of the coated particles is about −30, −25, −20, or −15 mV.

### 3. Methods of Encapsulating or Attaching Molecules to the Surface of Coated Particles

As introduced above, there are at least two principle groups of molecules to be encapsulated or attached to the particle, either directly or via a coupling molecule: targeting molecules and active agents e.g., therapeutic, nutritional, diagnostic or prophylactic agents. In some embodiments, the molecule is incorporated into the matrix of the layer, for example, by mixing the material with the polymer solution during manufacture of the coating. Additionally or alternatively, the materials can be coupled to one or more elements of the coating using standard techniques. For example, the targeting molecule or therapeutic molecule to be delivered can be coupled directly to the polymer or to a material such as a fatty acid which is incorporated into the polymer.

Functionality refers to conjugation of a ligand to the surface of the particle via a functional chemical group (carboxylic acids, aldehydes, amines, sulfhydryls and hydroxyls) present on the surface of the particle and present on the ligand to be attached. Functionality may be introduced into the particles in two ways.

The first is during the preparation of the particles, for example during the emulsion preparation of particles by incorporation of stabilizers with functional chemical groups.

A second is post-particle preparation, by direct crosslinking particles and ligands with homo- or heterobifunctional crosslinkers. This second procedure may use a suitable chemistry and a class of crosslinkers (CDI, EDAC, glutaraldehydes, etc. as discussed in more detail below) or any other crosslinker that couples ligands to the particle surface via chemical modification of the particle surface after preparation. This second class also includes a process whereby amphiphilic molecules such as fatty acids, lipids or functional stabilizers may be passively adsorbed and adhered to the particle surface, thereby introducing functional end groups for tethering to ligands.

Various other methods of producing a nanocarriers encapsulating an active agent are known in the art. For example, methods of loading nanoporous structures are reviewed in Wang et al., J. Mater. Chem. 19, 6451 (2009). In one example, nanocarriers may be loaded by contacting the nanocarrier with an aqueous solution of an active followed by a period of incubation. The active solution can contain an excess of the amount of active to be loaded onto the supraparticle and incubation can occur at room temperature. Agitation of the solution containing the supraparticle and the payload may be used to enhance loading of the payload.

### III. Formulations

Pharmaceutical compositions including the disclosed iodide particles, for example KI nanoparticles, are provided. Pharmaceutical compositions can be for, for example, administration by parenteral (e.g., intramuscular, intraperitoneal, intravenous (IV), intrathecal, or subcutaneous) injection.

In some embodiments, the compositions are administered systemically, for example, by intravenous or intraperitoneal administration, in an amount effective for delivery of the compositions to targeted cells.

In certain embodiments, the compositions are administered locally, for example, by subcutaneous injection, or injection directly into a site to be treated. In some embodiments, the compositions are injected or otherwise administered directly to one or more tumors. Typically, local injection causes an increased localized concentration of the compositions which is greater than that which can be achieved by systemic administration, and/or may reduce toxicity to other tissues (e.g., non-tumor cells). In some embodiments, the compositions are delivered locally to the appropriate cells by using a catheter or syringe. Other means of delivering such compositions locally to cells include using infusion pumps (for example, from Alza Corporation, Palo Alto, Calif.) or incorporating the compositions into polymeric implants (see, for example, P. Johnson and J. G. Lloyd-Jones, eds., Drug Delivery Systems (Chichester, England: Ellis Horwood Ltd., 1987), which can effect a sustained release of the particles to the immediate area of the implant.

In some embodiments, the particle compositions are intravesically administered to the bladder. Such a method of delivery is particularly useful for the treat bladder cancer.

The iodide particles, for example KI nanoparticles, can be provided to the cell either directly, such as by contacting it with the cell, or indirectly, such as through the action of any biological process. For example, the iodide particles, for example KI nanoparticles, can be formulated in a physiologically acceptable carrier or vehicle, and injected into a tissue or fluid surrounding the cell.

### A. Formulations for Parenteral Administration

In a preferred embodiment the compositions are administered in an aqueous solution, by parenteral injection.

The formulation can be in the form of a suspension or emulsion. In general, pharmaceutical compositions are provided including effective amounts of iodide particles, for example KI nanoparticles, optionally include pharmaceutically acceptable diluents, preservatives, solubilizers, emulsifiers, adjuvants and/or carriers. Such compositions can include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions., or by heating the compositions.

In some embodiments, increasing temperature of a colloidal solution of iodide particles is avoided. In some embodiments, lipid iodide nanoparticles can be prepared in a thin film, which can optionally undergo heating. For example, phospholipid can be mixed with nanoparticles in organic solvents such as chloroform. After evaporating chloroform, a thin film is left on the vessel interior surface. Nanoparticles can be shipped in this manner. Before treatment, water/buffer solutions are added to the vessel to redisperse nanoparticles in aqueous solutions.

### B. Other Formulations

The iodide particles, for example KI nanoparticles, can also be applied topically. Topical administration can include application to the lungs, nasal, oral (sublingual, buccal), vaginal, or rectal mucosa. These methods of administration can be made effective by formulating the iodide particles, for example KI nanoparticles, with transdermal or mucosal transport elements. In particular embodiments, the route of administration is nasal administration.

A wide range of mechanical devices designed for pulmonary delivery of therapeutic products can be used, including but not limited to, nebulizers, metered dose inhalers, and powder inhalers, all of which are familiar to those skilled in the art. Some specific examples of commercially available devices are the Ultravent® nebulizer (Mallinckrodt Inc., St. Louis, Mo.); the Acorn® II nebulizer (Marquest Medical Products, Englewood, Colo.); the Ventolin® metered dose inhaler (Glaxo Inc., Research Triangle Park, N.C.); and the Spinhaler® powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa can be incorporated into a tablet, gel, capsule, suspension or emulsion. Standard pharmaceutical excipients are available from any formulator.

Oral formulations may be in the form of chewing gum, gel strips, tablets, capsules, or lozenges. Oral formulations may include excipients or other modifications to the particle which can confer enteric protection or enhanced delivery through the GI tract, including the intestinal epithelia and mucosa (see Samstein, et al., Biomaterials, 29 (6): 703-8 (2008).

Transdermal formulations may also be prepared. These will typically be ointments, lotions, sprays, or patches, all of which can be prepared using standard technology. Transdermal formulations can include penetration enhancers.

### IV. Methods of Use

### A. Methods of Treatment

Iodine has shown promise in enhancing radiotherapy (Tamura, et al., Scientific reports, 7, 43667 (2017), Wang, et al., European journal of radiology, 92, 72-77 (2017), Obeid, et al., Journal of Cerebral Blood Flow & Metabolism, 34 (4), 638-645 (2014)). However, conventional iodine compounds show fast clearance and low retention inside cancer cells, limiting their application as a radiosensitizer. The Examples below provide experiments support use of PMAO-KI NPs as a radiosensitizer for RT in breast cancer. Potassium iodide (KI) NP-based particles are believed to employ the Na⁺/I⁻ symporter (NIS) for iodine delivery. NIS is a transmembrane protein that co-transports two Na⁺ and an I⁻ across the cellular membrane. NIS is mainly expressed on thyroid cells for uptake and incorporation of iodine for hormone synthesis (Lazar, et al., The Journal of Clinical Endocrinology & Metabolism, 84 (9), 3228-3234 (1999), Spitzweg, et al., The Journal of Clinical Endocrinology & Metabolism, 86 (7), 3327-3335 (2001), Arroyo-Helguera, et al., Endocrine-related cancer, 13 (4), 1147-1158 (2006)). Recent analysis showed that NIS is also positive in 76-87% of human invasive breast cancers, including triple negative cases (Daniels & Haber, Nature medicine, 6 (8), 859 (2000), Wapnir, et al., The Journal of Clinical Endocrinology & Metabolism, 88 (4), 1880-1888 (2003)). The experimental results below show remarkable improvement in tumor suppression was observed among animals receiving i.t. injection of PMAO-KI NPs before irradiation.

Additionally, radioisotopes such as ¹³¹I and ¹²⁵I can incorporated into iodide particles, producing “hot” particles that can serve as a radiopharmaceutical or a brachytherapy agent for cancer treatment. In addition, radioisotopes such as ¹²³I and ¹²⁴I can be incorporated into iodide particles, and the resulting nanoparticles can serve as imaging probes for tumor diagnosis.

Targeting to particular cell types, including, but not limited to, tumor cells, can be enhanced by the addition of a targeting agent, and/or the functional activity can be enhanced by the addition of an active agent such as a therapeutic active agent designed to enhance tumor cell death.

For example, NPs can be systemically administered (for instance intravenously injected, etc.), and the nanoparticles can accumulate in tumors through either the enhanced permeability and retention (EPR) effect (i.e., passive targeting) or ligand-receptor interaction (e.g., active targeting). Iodine or radioiodine can be released from the nanoparticles and enter cancer or other target cells through NIS.

Thus, methods of treating cancer are provided.

In some methods, iodide particles are administered in an effective amount to enhance radiotherapy and/or provide a direct anti-tumor effect (e.g., tumor cell death). In the experiments below, PMAO-KI NPs were intratumarally injected in mice at 50 μg/mL in 50 μL PBS (e.g., about 0.1-0.2 mg/kg). As further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired.

Generally dosage levels of 0.001 to 10 mg/kg, for example, 0.05 mg/kg to 1 mg/kg, of body weight are administered to mammals. Generally, for local administration, dosage may be lower than for systemic administration. The dosage can be a daily dosage, or any other dosage regimen consistent with the disclosed methods. The timing of the administration of the composition will also depend on the formulation and/or route of administration used. The compound may be administered once daily, but may also be administered two, three or four times daily, or every other day, or once or twice per week. For example, the subject can be administered one or more treatments 1, 2, 3, 4, 5, 6, 7, 8, 9, 10, 11, 12, 13, 14, 15, 16, 17, 18, 19, 20, 21, 22, 23, or 24 hours, days, weeks, or months apart.

The subject can have one or more malignant or non-malignant tumors. In some embodiments, the subject has cancer.

### 1. Radiosensitizing Applications

In some embodiments, the composition is administered to the subject as a radiosensitizer in combination with a radiation therapy. Although discussed herein primarily with reference to ionizing radiation therapy, it is believed that the disclosed compounds may also be used as sensitizers in proton therapy. Thus, substitution of ionizing radiation with proton therapy in the methods disclosed herein are specifically contemplated and disclosed.

In preferred radiosensitizing embodiments, the particles are KI nanoparticles optionally with radioisotopic iodide, and optionally but preferably a coating, e.g., a polymeric coating.

In some embodiments, the iodide nanoparticles are administered in an effective amount to enhance treatment of the tumor or cancer relative to administration of radiation alone, and/or administration of iodide nanoparticles alone. Enhance treatment of the tumor can include, for example, a greater or improved effect with the same dose and/or frequency of radiation treatments, or the same or similar effects with a lower dose and/or reduced frequency of radiation treatments. In some embodiments, the administration of the iodide nanoparticles has reduced systemic toxicity and/or greater in vitro and/or in vivo half-life compared to administration of a free iodide or other non-particle-based iodide-containing compound such as Iomeprol.

In preferred embodiments, the radiation is ionizing radiation. Ionizing radiation therapy (also referred to as radiotherapy and RT) is the medical use of ionizing radiation as part of cancer treatment to control malignant cells. Ionizing radiation is typically defined as radiation with enough energy to liberate an electron from the orbit of an atom, causing the atom to become charged or ionized. Ionizing radiation can be administered to a subject in need thereof as part of radiation therapy for the treatment for cancer. Examples of radiation therapy include, but are not limited to, external beam radiation therapy (EBRT or XRT) or teletherapy, brachytherapy or sealed source radiation therapy, and systemic radioisotope therapy or unsealed source radiotherapy. The radiation therapy can be administered to the subject externally (i.e., outside the body), or internally for example, brachytherapy which typically utilizes sealed radioactive sources placed in the area under treatment, and or systemic administration of radioisotopes by infusion or oral ingestion. Radiation therapy can include temporary or permanent placement of radioactive sources on or within the subject. Another example of radiation therapy is particle therapy which is typically includes external beam radiation therapy where the particles are protons or heavier ions.

Radiation therapy works by damaging the DNA of dividing cells, e.g., cancer cells. This DNA damage is caused by one of two types of energy, photon or charged particle. This damage is either direct or indirect. Indirect ionization happens as a result of the ionization of water, forming free radicals, notably hydroxyl radicals, which then damage the DNA. For example, most of the radiation effect caused by photon therapy is through free radicals. One of the major limitations of photon radiotherapy is that the cells of solid tumors become deficient in oxygen, and tumor cells in a hypoxic environment may be as much as 2 to 3 times more resistant to radiation damage than those in a normal oxygen environment.

Direct damage to cancer cell DNA occurs through high-LET (linear energy transfer) charged particles such as proton, boron, carbon or neon ions. This damage is independent of tumor oxygen supply because these particles act mostly via direct energy transfer usually causing double-stranded DNA breaks. Due to their relatively large mass, protons and other charged particles have little lateral side scatter in the tissue; the beam does not broaden much, stays focused on the tumor shape and delivers small dose side-effects to surrounding tissue.

The amount of radiation used in photon radiation therapy is measured in Gray (Gy), and varies depending on the type and stage of cancer being treated. For curative cases, the typical dose for a solid epithelial tumor ranges from 60 to 80 Gy, while lymphomas are treated with 20 to 40 Gy. Post-operative (adjuvant) doses are typically around 45-60 Gy in 1.8-2 Gy fractions (for breast, head, and neck cancers). Many other factors are considered by radiation oncologists when selecting a dose, including whether the patient is receiving chemotherapy, patient co-morbidities, whether radiation therapy is being administered before or after surgery, and the degree of success of surgery.

The response of a cancer to radiation is described by its radiosensitivity. Highly radiosensitive cancer cells are rapidly killed by modest doses of radiation. These include leukemias, most lymphomas and germ cell tumors. The majority of epithelial cancers are only moderately radiosensitive, and require a significantly higher dose of radiation (60-70 Gy) to achieve a radical cure. Some types of cancer are notably radioresistant, that is, much higher doses are required to produce a radical cure than may be safe in clinical practice. Renal cell cancer and melanoma are generally considered to be radioresistant.

In some embodiments, the compositions and methods reduce the dose of radiation required to induce a curative or preventative effect. For example, the disclosed compounds can increase a cancer's radiosensitivity. Effective doses of radiation therapy may be toxic for certain cancers. In some embodiments, the compounds decrease the required effective dose of radiation needed to treat a cancer, thereby reducing toxicity of the effective dose of radiation.

In other embodiments, the disclosed compounds may be used with normal doses of drug or radiation to increase efficacy. For example, the compounds may be used to potentiate a radiation therapy for a cancer that is radiation resistant.

The response of a tumor to radiotherapy is also related to its size. For complex reasons, very large tumors respond less well to radiation than smaller tumors or microscopic disease. Various strategies are used to overcome this effect. The most common technique is surgical resection prior to radiotherapy. This is most commonly seen in the treatment of breast cancer with wide local excision or mastectomy followed by adjuvant radiotherapy. Another method is to shrink the tumor with neoadjuvant chemotherapy prior to radical radiotherapy. In some embodiments, the disclosed methods allow for treatment of tumors that are larger than can be treated by a normal dose of radiation.

A third technique is to enhance the radiosensitivity of the cancer by giving certain drugs during a course of radiotherapy. The disclosed compositions can serve this third function. In these embodiments, the compound increases the cell's sensitivity to the radiotherapy, for example, by at least 10%, 15%, 20%, 25%, 30%, 35%, 40%, 45%, 50%. Moreover, the compound can be combined with one or more additional radiosensitizers. Examples of known radiosensitizers include paclitaxel, cyclophosphamide, cisplatin, gemcitabine, 5-fluorouracil, pentoxifylline, vinorelbine, PARP inhibitors, histone deacetylase inhibitors, proteasome inhibitors, and other mentioned elsewhere herein.

Radiation therapy can be administered to a subject in combination with surgery, chemotherapy, hormone therapy, immunotherapy, or combination thereof. For example, intraoperative radiation therapy or (IORT) is delivered immediately after surgical removal of a cancer. This method has been employed in breast cancer (TARGeted Introperative radiation therapy or TARGIT), brain tumors and rectal cancers.

Radiotherapy also has several applications in non-malignant conditions, such as the treatment of trigeminal neuralgia, severe thyroid eye disease, pterygium, pigmented villonodular synovitis, prevention of keloid scar growth, and prevention of heterotopic ossification. Thus, in some embodiments, the compositions and methods are used to increase radiosensitivity for a non-malignant condition.

Typically, the prodrug composition is administered before, e.g., minutes, hours, or days before, a radiation therapy. For example, in exemplary embodiments, a dose of radiation is administered 0 hour to 48 hours, or 0.5 hour to 24 hours, or 1 hour to 12 hours, or 1 hour to 6 hours, or 2 hours to 6 hours, or any subrange or specific integer or fraction thereof therebetween, for example 0 (i.e., contemporaneously or concurrently with), or 0.25, 0.5, 0.75, 1, 2, 3, 4, or 5 hours after administration of the pharmaceutical composition. In some embodiments, 1, 2, 3, 4, or 5 rounds are radiation are administered after each single dose of the iodide nanoparticles. In some embodiments, the iodide nanoparticles are administered one or more times for each round of radiation. In some embodiments, each cycle of radiation is preceded by a cycle of iodide nanoparticles. For example, in particular embodiments, 1, 2, 3, 4, 5, 6, 7, 8, 9, 10, or more rounds of administration of the pharmaceutical composition followed by administration of the dose of radiation are carried out in tandem.

In some embodiments, the disclosed compositions and methods are more effective, less toxic, or combination thereof relative to a specific concurrent or sequential chemo-radiotherapy (CRT).

### 2. Direct Anti-tumor Applications

In some embodiments, particularly wherein the nanoparticles include a radioisotope such as ¹³¹I and ¹²⁵I, and/or where a therapeutic active agent is incorporated into the particles (e.g., as part or attached to a coating or layer over the iodide core the compositions can be administered for therapeutic effect. Although secondary radiotherapy, e.g., external beam radiation, can be administered in combination with these particles, it is not required because the particles themselves serve a source of radiotherapy and/or delivery means of another therapeutic agent. Thus, in some embodiments, iodide nanoparticles are administered to a subject in need thereof in an effective amount to treat cancer, in the absence co-administration of radiotherapy (or if there is a radioisotope such ¹³¹I and ¹²⁵I incorporated into the particle, a second radiotherapy).

### B. Methods of Imaging and Diagnosis

The disclosed compositions can also be used for imaging. Such methods typically include administering a subject an effective amount of iodide nanoparticles typically including a radioisotope.

It will be understood in the art that the size of the subject and the imaging system used will determine the quantity of imaging moiety needed to produce diagnostic images. Depending on several variables, including the type of label used and the mode of administration, the time interval following the administration for permitting the labeled molecule to preferentially concentrate at sites in the subject and for unbound labeled molecule to be cleared to background level can be, e.g., 6 to 48 hours or 6 to 24 hours or 6 to 12 hours. In another embodiment the time interval following administration is 1 to 20 days or 5 to 10 days.

Such methods can be used as part of diagnostic or prognostic method. In some embodiments, monitoring of a disease or disorder is carried out by repeating the method for diagnosing the disease or disorder, for example, one month after initial diagnosis, six months after initial diagnosis, one year after initial diagnosis, etc. Similarly, the methods can be used to monitor the efficacy or effectiveness of a treatment or intervention, by, e.g., taking images before and after one or more treatments or interventions.

The presence of the labeled molecule can be detected in the subject using methods known in the art as in vivo scanning. These methods depend upon the type of label used. Skilled artisans will be able to determine the appropriate method for detecting a particular label. Methods and devices that may be used in the diagnostic methods of the disclosure include, but are not limited to, position emission tomography (PET) and single-photon emission computerized tomography (SPECT). Thus, in particular embodiments, the isotope is ¹²³I or ¹²⁴I, and the subject is imaged by PET and or SPECT. Typically, ¹²³I is a radioisotope used for SPECT imaging, and ¹²⁴I is used for PET imaging.

As with the methods of treatment, administration may be as discussed in more detail elsewhere herein, e.g., systemic or local to the site of interest.

### C. Cancers to be Treated and Imaged

The compositions and methods described herein are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth. The treatment is also useful for reducing overproliferation of non-cancerous tissues such as endometriosis, restenosis, and scarring (fibrosis).

Malignant tumors which may be treated are classified according to the embryonic origin of the tissue from which the tumor is derived. Carcinomas are tumors arising from endodermal or ectodermal tissues such as skin or the epithelial lining of internal organs and glands. The disclosed compositions are particularly effective in treating carcinomas. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The types of cancer that can be treated with the provided compositions and methods include, but are not limited to, cancers such as vascular cancer such as multiple myeloma, adenocarcinomas and sarcomas, of bone, bladder, brain, breast, cervical, colo-rectal, esophageal, kidney, liver, lung, nasopharangeal, pancreatic, prostate, skin, stomach, uterine, and combination thereof. In some embodiments, the disclosed compositions are used to treat multiple cancer types concurrently. Additionally or alternatively, the compositions and methods can be used to treat metastases, including but not limited to metastases of any of the cancers discussed herein. The compositions can also be used to treat metastases or tumors at multiple locations.

In some embodiments, the cells that are the target of treatment, e.g., malignant or non-malignant tumor cells express or overexpress the Na+/I-symporter (NIS).

In some embodiments the cancer is breast cancer. NIS is detected in mammary gland, thus, in particular embodiments the breast cancer cells express or overexpress the NIS. In some embodiments, the breast cancer is or includes Ductal Carcinoma In Situ (DCIS), Invasive Ductal Carcinoma (IDC), IDC Type: Tubular Carcinoma of the Breast, IDC Type: Medullary Carcinoma of the Breast, IDC Type: Mucinous Carcinoma of the Breast, IDC Type: Papillary Carcinoma of the Breast, IDC Type: Cribriform Carcinoma of the Breast, Invasive Lobular Carcinoma (ILC), Inflammatory Breast Cancer, Lobular Carcinoma In Situ (LCIS), Male Breast Cancer, Molecular Subtypes of Breast Cancer, Triple-Negative Breast Cancer, Paget's Disease of the Nipple, Phyllodes Tumors of the Breast, Recurrent Breast Cancer, and/or Metastatic Breast Cancer. The breast cancer can be estrogen receptor (ER) positive or negative, progesterone receptor (PR) positive or negative, and/or hormone receptor (HR) positive or negative. The breast cancer can be HER2 positive and/or positive for one or more other tumor markers. Thus, in some embodiments, the breast cancer is ER positive and PR positive, but negative for HER2 (Group 1 (luminal A)); ER positive, PR negative and HER2 positive (Group I, (luminal B)); ER negative and PR negative, but HER2 positive (Group 3 (HER2 positive)); or triple-negative breast cancer, which includes tumors that are ER negative, PR negative and HER2 negative (Group 4 (basal-like)).

NIS also mediates active iodide transport into the thyroid gland and several extra-thyroidal tissues, where iodine serves as the basis for thyroid hormone biosynthesis (Yao, et al., Oncol Rep, 34:59-66, (2015)). Thus, in some embodiments, the disclosed compositions and methods are used to treat hyperthyroidism and/or thyroid cancer, optionally, but preferably where the target cells express or overexpress NIS. NIS is also detected in placenta, salivary and digestive gland, and thus, in some embodiments, cells of the placenta, salivary and digestive gland, or tumors formed therefrom, are the target of the disclosed compositions and methods.

In some embodiments, the cancer is a highly radiosensitive, moderately radiosensitive cancer, or radioinsensitive (i.e., low radiosensitive cancer). Highly radiosensitive cancer cells are rapidly killed by modest doses of radiation. Tissues rich in actively dividing cells generally show high sensitivity to radiation, whereas those with few such cells have low radiosensitivity (Hayabuchi, JMAJ, 47 (2): 79-83 (2004)). For example, genital glands such as the testis and ovary, lymphatic tissue, fetal tissue, and fetus-like blast cell tissue are highly radiosensitive. Tissues with low radiosensitivity include adult bone, fatty tissue, muscle, and large vessels. Because the radiosensitivity of a tumor reflects the sensitivity of the tissue from which it has arisen, malignant lymphomas, which originate in lymphatic tissue, and seminomas, which originate in the testis, have high sensitivity to radiation. In contrast, osteogenic sarcomas and liposarcomas demonstrate low radiosensitivity.

Epithelial tumors and cancers, are considered to have moderate radiosensitivity. Such cancers can require a significantly higher dose of radiation (60-70 Gy) to achieve a cure. Among these tumors, undifferentiated carcinoma and small cell carcinoma have relatively high radiosensitivity, followed by squamous cell carcinoma. The radiosensitivity of adenocarcinoma is generally lower than that of other types of epithelial tumors. In light of this, head and neck cancer, esophageal cancer, uterine cervical cancer, and skin cancer, among which squamous cell carcinoma is common, seem to be good indications for radiotherapy.

However, even among squamous cell carcinomas of the esophagus, some are highly radiosensitive but others are not. Radiosensitivity can depend not only on the histologic type of the tumor but also on other factors, for example, the oxygen concentration in the tumor and the mitotic cycle of tumor cells.

Renal cell cancer and melanoma are generally considered to be radioresistant.

In other particular embodiments, the cancer is a lung cancer, a colon cancer, and/or metastasis thereof.

In some embodiments, the methods include taking action to promote

NIS expression in the target cells or tissue of the treatment, e.g., tumor cells to enhance iodine uptake. To promote NIS expression in target tissue such as tumors, trans retinoic acid (tRA) can be administered before iodide nanoparticle injection. tRA can be administered orally, intravenously, or intratumorally. The dosage is typically one that is effective to increase NIS on the target cells or tissue. In some embodiments, NIS is not increased or is minimally increased on non-target cells. In some embodiments, the injection dose is about 45-90 mg/m². Iodide nanoparticles can be administered concurrently or after tRA injection, for example, 0-48 hours after nanoparticle administration.

### D. Combination Therapy

In some embodiments, the methods include administration of iodide particles in combination with one or more additional active agents. The additional active agent can be, for example, a traditional therapy for the disease or disorder being treated.

The term “combination” or “combined” is used to refer to either concomitant, simultaneous, or sequential administration of two or more active agent compounds. The combinations can be administered either concomitantly (e.g., as an admixture), separately but simultaneously (e.g., via separate intravenous lines into the same subject; one agent is given orally while the other agent is given by infusion or injection, etc.,), or sequentially (e.g., one agent is given first followed by the second).

Exemplary active agents include, for example, chemotherapeutics, especially antineoplastic drugs. The majority of chemotherapeutic drugs can be divided into alkylating agents, antimetabolites, anthracyclines, plant alkaloids, topoisomerase inhibitors, monoclonal antibodies, and other antitumor agents. In particular embodiments, the additional active agent is an alkylating agent (such as temozolomide, cisplatin, carboplatin, oxaliplatin, mechlorethamine, cyclophosphamide, chlorambucil, dacarbazine, lomustine, carmustine, procarbazine, chlorambucil and ifosfamide), an antimetabolite (such as fluorouracil, gemcitabine, methotrexate, cytosine arabinoside, fludarabine, and floxuridine), anantimitotic or vinca alkaloid (such as vincristine, vinblastine, vinorelbine, and vindesine), an anthracycline (including doxorubicin, daunorubicin, valrubicin, idarubicin, and epirubicin, as well as actinomycins such as actinomycin D), a cytotoxic antibiotic (including mitomycin, plicamycin, and bleomycin), or a topoisomerase inhibitor (including camptothecins such as irinotecan and topotecan and derivatives of epipodophyllotoxins such as amsacrine, etoposide, etoposide phosphate, and teniposide).

The invention can be further understood through the following numbered paragraphs.

1. A nanoparticle formed from iodide and ions of an alkali metal or alkaline earth metal.

2. The nanoparticle of paragraph 1, wherein the alkali metal is selected from potassium, lithium, sodium, rubidium, and cesium, and the alkaline earth metal is selected from magnesium and calcium.

3. The nanoparticle of paragraph 2, wherein the alkali metal is potassium.

4. A nanoparticle formed from iodide and potassium ions.

5. The nanoparticle of any one of paragraphs 1-4, comprising a radioisotope.

6. The nanoparticle of paragraph 5, wherein the radioisotope is ¹³¹I, ¹²⁵I, ¹²⁴I, or ¹²³I.

7. The nanoparticle of paragraph 6, wherein the iodine is, or is formed from, the ¹³¹I, ¹²⁵I, ¹²⁴I, or ¹²³I.

8. The nanoparticle of any one of paragraphs 1-7, wherein the nanoparticle is cubic.

9. The nanoparticle of any one of paragraphs 1-8, wherein the nanoparticle is between about 15 nm and about 800 nm, or between about 20 nm and about 500 nm, or between about 50 nm and about 350 nm, or about 50 nm to about 125 nm, or any subrange or specific integer there between.

10. The nanoparticle of paragraph 9, wherein the nanoparticle is about 80 nm.

11. The nanoparticle of any one of paragraphs 1-10, further comprising a coating.

12. The nanoparticle of paragraph 11, wherein the coating comprises one or more polymers, peptides, proteins, lipids, or a combination thereof.

13. The nanoparticle of paragraphs 11 or 12, wherein the coating comprises poly(maleic anhydride alt-1-octadecene).

14. The nanoparticle of any one of paragraphs 11-13, wherein the coating comprises a thickness of 1 nm to 200 nm, or 10 nm to 100 nm, or 25 nm to 75 nm inclusive, or any subrange or specific integer therebetween.

15. The nanoparticle of paragraph 14, wherein the coating has a thickness of about 50 nm.

16. The nanoparticle of any one of paragraphs 1-15, comprising a targeting moiety.

17. The nanoparticle of paragraph 16, wherein the targeting moiety targets a cell surface or transmembrane protein.

18. The nanoparticle of paragraphs 16 or 17, wherein the targeting moiety targets a cell specific marker.

19. The nanoparticle of any one of paragraphs 16-18, wherein the targeting moiety targets a tumor associated or cancer antigen.

20. The nanoparticle of any one of paragraphs 16-19, wherein the targeting moiety targets the sodium/iodide symporter (NIS).

21. The nanoparticle of any one of paragraphs 16-20, wherein the targeting moiety is a ligand, receptor, antibody, or aptamer.

22. The nanoparticle of any one of paragraphs 16-21, wherein the targeting moiety is incorporated into the coating, optionally wherein the targeting moiety is conjugated to a component of the coating.

23. The nanoparticle of any one of paragraphs 1-22 comprising an active agent.

24. The nanoparticle of paragraph 23, wherein the active agent is selected from therapeutic, nutritional, diagnostic, and prophylactic agents.

25. The nanoparticle of paragraph 24, wherein the active agent is a chemotherapeutic agent.

26. The nanoparticle of any one of paragraphs 23-25, wherein the active agent is incorporated into the coating, optionally wherein the active agent is conjugated to a component of the coating.

27. A pharmaceutical composition comprising a plurality of the nanoparticles of any one of paragraphs 1-26.

28. The pharmaceutical composition of paragraph 27, wherein the average hydrodynamic size of the nanoparticles is between about 15 nm and about 800 nm, or between about 20 nm and about 500 nm, or between about 50 nm and about 350 nm, or about 50 nm to about 125 nm, or any subrange or specific integer there between ±5%, 10%, 15%, 20%, or 25%.

29. The pharmaceutical composition of paragraphs 27 or 28, wherein the nanoparticles are monodisperse.

30. A method of making potassium iodide nanoparticles comprising reacting potassium oleate with I₂ in octadecene in the presence of oleylamine.

31. The method of paragraph 30 further comprising adding a coating to the nanoparticles.

32. The method of paragraph 31, wherein the coating comprises poly(maleic anhydride alt-1-octadecene).

33. A method of sensitizing a subject to radiation therapy comprising administering to a subject in need thereof the pharmaceutical composition comprising an effective amount of the nanoparticle of any one of paragraphs 1-26.

34. A method of treating a subject for cancer comprising sensitizing the subject to radiation therapy according to method of paragraph 33 and administering the subject one or more doses of radiation therapy.

35. The method of paragraph 34, wherein the radiation therapy is ionizing radiation therapy, phototherapy, or proton therapy.

36. The method of paragraphs 34 and 35, wherein the pharmaceutical composition enhances the treatment of the cancer compared to administration of the radiation alone.

37. The method of any one of paragraphs 34-36, wherein the cancer is a radiosensitive cancer.

38. The method of any one of paragraphs 34-36, wherein the cancer is a radioresistant cancer.

39. The method of any one of paragraphs 34-38, wherein the same dose of radiation is more effective than when administered in the absence of the pharmaceutical composition, a lower dose of radiation has the same effectiveness as a higher dose when administered in the absence of the pharmaceutical composition, or a combination thereof.

40. The method of any one of paragraphs 34-39, wherein a dose of radiation is administered after administration of the pharmaceutical composition.

41. The method of any one of paragraph 34-40, wherein the dose of radiation is administered 0 hour to 48 hours, or 0.5 hour to 24 hours, or 1 hour to 12 hours, or 1 hour to 6 hours, or 2 hours to 6 hours, or any subrange or specific integer or fraction thereof therebetween, for example 0 (i.e., contemporaneously with), or 0.25, 0.5, 0.75, 1, 2, 3, 4, or 5 hours after administration of the pharmaceutical composition.

42. The method of any one of paragraphs 34-41, comprising 1, 2, 3, 4, 5, 6, 7, 8, 9, 10, or more rounds of administration of the pharmaceutical composition followed by administration of the dose of radiation.

43. The method of any one of paragraphs 34-42, wherein the radiation is ionizing radiation.

44. A method of treating a subject for cancer comprising administering the subject an effective amount a pharmaceutical composition comprising an effective amount of the nanoparticle of any one of paragraphs 1-26, wherein the nanoparticle comprises a radioisotope or an anti-cancer active agent.

45. A method of imaging a subject in need thereof comprising administering the subject an effective amount a pharmaceutical composition comprising an effective amount of the nanoparticle of any one of paragraphs 1-26, wherein the nanoparticle comprises a radioisotope, and detecting the radioisotope, optionally wherein the subject has cancer.

46. The method of paragraph 45, wherein the subject has cancer.

47. The method of paragraph 44, wherein the nanoparticle comprises the radioisotope ¹³¹I or ¹²⁵I.

48. The method of paragraph 45, wherein the nanoparticle comprises the radioisotope ¹²⁴I or ¹²³I.

49. The method of paragraph 45, wherein the radioisotope is detected by positron emission tomography (PET) imaging, optionally wherein the radioisotope is ¹²⁴I.

50. The method of paragraph 45, wherein the radioisotope is detected by single-photon emission computerized tomography (SPECT) imaging, optionally wherein the radioisotope is ¹²³I.

51. The method of any one of paragraphs 34-50, wherein the cancer is a vascular, bone, muscle, bladder, brain, breast, cervical, colo-rectal, esophageal, kidney, liver, lung, nasopharangeal, pancreatic, prostate, skin, stomach, uterine, or germ cell cancer.

52. The method of paragraph 51, wherein cells of the cancer express the Na+/I-symporter (NIS).

53. The method of any one of paragraphs 34-52, wherein the cancer is a breast cancer.

54. The method of any one of paragraphs 34-52, wherein the cancer is a thyroid cancer.

55. The method of any one of paragraphs 33-54, wherein the pharmaceutical composition is administered by injection or infusion locally to the site in need of treatment, optionally wherein the site is a tumor.

### EXAMPLES

### Example 1: Fabrication and Characterization of PI Nanoparticles

### Materials and Methods

### Potassium Iodide Nanoparticle (NP) Synthesis.

6.68 g of potassium oleate (40 wt. % paste in H₂O Sigma-Aldrich, 291242) was added to a round bottom flask with 200 mL 1-Octadecene (Sigma-Aldrich, 0806, 90%), and mixed at 290° C. until oleate was fully solubilized. Once the reaction is cool 10 mL of Oleylamine (Sigma-Aldrich, O7805, 70%) is added and mixed. After, 5 g of Iodine (Sigma-Aldrich, 207772) is added and the reaction is sealed and mixed overnight. Then 8 aliquots of NPs are centrifuged at 12096×g for 10 mins. The particles are then washed three times in EtOH and placed in an oven until dry.

### PMAO Coating.

Previously synthesized KI NPs in 3 mL of chloroform was added to the mixture and sonicated into suspension. After which 120 mg Poly(maleic anhydride alt-1-octadecene) (Sigma-Aldrich, 776866) in 3 mL chloroform (Sigma-Aldrich, C2432) and 1 mL PEG-bis-amine (43 mg in 10 mL chloroform) (Sigma-Aldrich, P9906) were added. The reaction was then mixed for 2 hrs. The solution was then poured into a round bottom flask. The solvent was removed under a rotary evaporator and gentle heat (<40° C.) until dry. 1.5 mL bis-hexamethyl triamine (43 mg in 10 mL chloroform) (Fisher Scientific, B181425G) was added to the flask and sonicated until the film was resuspended. The solution was then placed under Rotovap again until dry at room temperature. 50 mM Borate buffer is then added to cover film and sonicated until film is fully in solution. After which the solution is filtered to remove excessive aggregates then centrifuged at 10,000 rpm for 10 minutes. After which and the supernatant is discarded and the precipitate dried below 120° C. to avoid polymer softening. The dried and sealed PMAO-KI NPs can be stored for at least 6 months with no discernable loss in quality.

### Characterization of KI NPs.

KI in ethanol were dried on formvar TEM grids at 50° C. overnight for TEM and STEM imaging. TEM images were acquired on a 120 kV HT7830 and a 300 kV high resolution H9500 TEM (Clemson Electron Microscopy Facility). SEM images and elemental mapping were taken on a FEI Teneo operating at 15 kV for images and 30 kV for elemental mapping (Georgia Electron Microscopy Facility). For release profile determination, NPs both coated and uncoated were suspended in PBS and transferred to a molecular weight cutoff 10,000 kDa (Slide-A-Lyzer Thermo Scientific). The filtrate was sampled at multiple timepoints over 72 hrs and the residual collected as well. The iodine content was then calculated based on an iodine electrode (perfectION™ Mettler Toledo). IR spectra was taken from the dried powder of NPs on a Nicolet iS10 FT-IR spectrometer. Dynamic light scattering of both coated and uncoated particles was performed using a Malvern Zetasizer Nano S90 and zeta potential measured with the same.

### Results

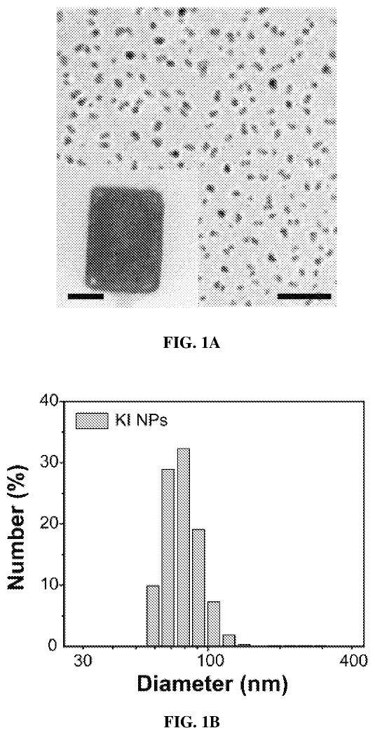

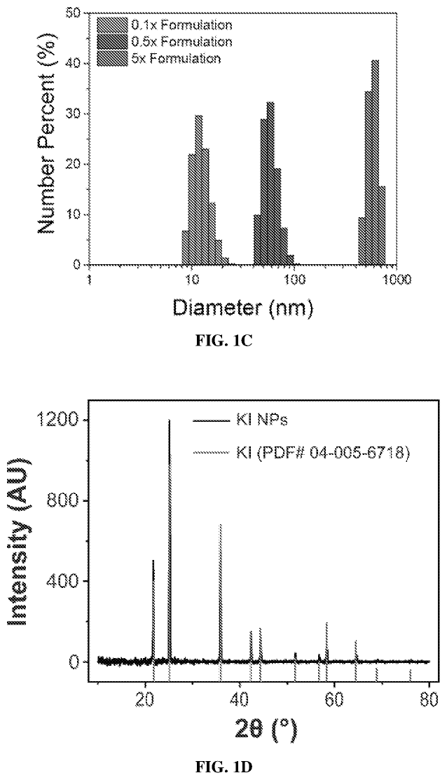

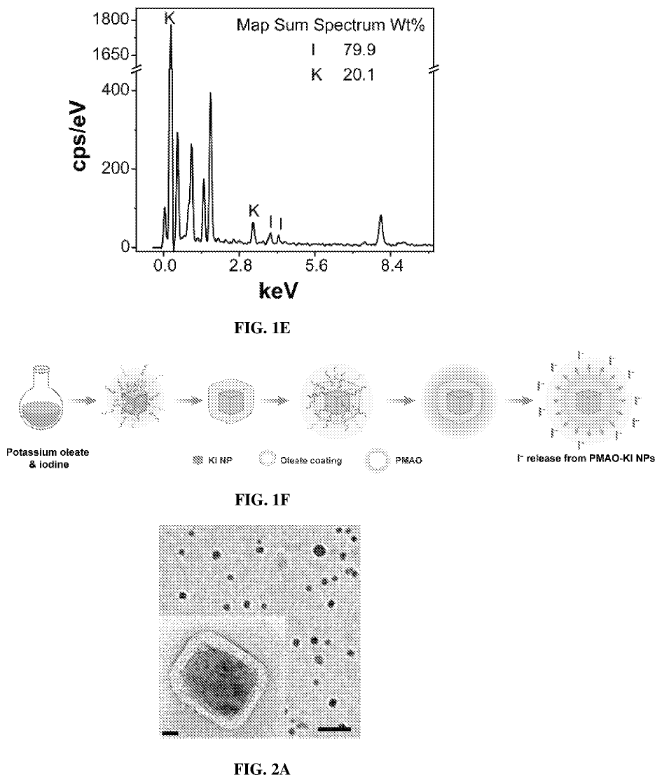

For nanoparticle synthesis, potassium oleate reacts with I₂ in octadecene in the presence of oleylamine at room temperature. It is postulated that oleate and oleylamine function in collaboration to provide reverse micelle structures, within which KI nucleates and forms particles (Sun, et al., science 2000, 287 (5460), 1989-1992 (2000)). Oleylamine also functions as a mild reducing agent to convert I₂ to I⁻ (Xu, et al., Chemistry of Materials, 21 (9), 1778-1780 (2009)). Typical reactions yielded monodisperse KI NPs with a diameter of 79.8±1.7 nm based on transmission electron microscopy (TEM, FIG. 1A). Dynamic light scattering (DLS) found a similar hydrodynamic size (FIG. 1B). By adjusting precursor amounts of potassium oleate and I₂, KI NPs in the range of 20-500 nm can be produced (FIG. 1C). X-Ray diffraction (XRD), as well as scanning electron microscopy (SEM) with energy dispersive X-ray analysis (EDXA), confirmed that the particles were KI in composition (FIG. 1D, 1E).

KI NPs were then coated with a layer of PMAO to be rendered with water solubility (FIG. 1F, FIG. 2A). PMAO was selected as the coating for a variety of reasons, including its biodegradability, biocompatibility, and readiness to be coupled with other molecules (Karandikar, et al., Eds, 263-293 (2017)). Briefly, KI NPs were mixed with PMAO and PEG-NH₂ in chloroform and, after removing the solvent by rotovap, bis-hexamethyl triamine in borate buffer (pH 8.2) was added. The hydrophobic alkyl chains of PMAO interacted with the oleate/oleylamine coating (Mazumder, et al., J Nanomater, 1-17, (2009)). The anhydride rings opened in the basic solution and reacted with primary amine, resulting in polymer crosslinking and PEGylation (FIG. 1F). The amount of PMAO can be tuned to adjust the final coating thickness. The resulting NPs are readily dispersed in water. Fourier-transform infrared spectroscopy (FT-IR) found OH and NH peaks, confirming successful PEGylation (FIG. 2B). The zeta potential of the PMAO-KI NPs was-23.4 mV, which is attributed to multiple surface carboxyl groups generated from anhydride ring opening (FIG. 2C).

The KI cores remain largely intact throughout the surface modification (FIG. 2A), but slowly degraded in aqueous solutions. To investigate this, PMAO-KI NPs were loaded onto a dialysis cassette and iodide released from the NPs was evaluated using an iodide-selective electrode. The time it took for half of the iodine to be released, or t_{1/2}, is negatively correlated with the coating thickness. A formulation with a coating thickness of ˜50 nm (FIG. 2A, 2D) and a t_{1/2} of ˜24 hours (FIG. 2E) was selected for follow-up studies.

### Example 2: KI NPs can Enhance Radical Production Under Beam Irradiation In Vitro

### Materials and Methods

### ROS Production.

Initial ROS testing was done using methylene blue trihydrate (MP Biomedicals) at a concentration of 5 mM with 25 μg/mL NPs. Irradiation was delivered via a Mini-X X-Ray Tube (Ampek) 50 keV at a dose rate of approximately 20 cGy/minute and would remain consistent throughout experiments. Absorbance change was measured via SynergyMX. A similar approach was used for APF (3′-(p-aminophenylfluorescein), Hydroxyl Radical, Hypochlorite or Peroxynitrite Sensor) (Invitrogen, 36003), DHE (Dihydroethidium (Hydroethidine), a superoxide indicator) (Invitrogen, D11347) and SOSG (Singlet Oxygen Sensor Green, Invitrogen, S36002) following the kit manufacturing instructions with 50 μg/mL NPs.

### MTT Assays.

A MTT assay (Thiazolyl Blue Tetrazolium Bromide, Sigma, M2128) was used to estimate the cytotoxicity of PMAO-KI NPs and phosopholipid (DSPE-PEG (2000) Amine) coated KI NPs on MCF-7 cells as well as radiosensitizer efficacy. Cells were seeded in 96 well-plates (Corning, 3599) with 10,000 cells/well. 24 hrs after cellular seeding, cells were treated with PMAO-KI NPs and KI solution of equivalent iodine content at various concentrations (1 μg/mL to 400 μg/mL). The cellular media was replaced with media of the appropriate concentration of materials and incubated for a further 24 hours. MTT reagent was then added in the culture medium, followed by incubation at 37° C. for 3 h and the resulting purple crystal solubilized in DMSO. The absorbance was measured on a microplate reader (SynergyMx) at 590 nm, with the measurements performed in at least triplicate.

For X-ray therapy a similar protocol was followed. 24 hours after cellular seeding tRA in DMSO was added to the cell wells to a final tRA concentration of 1 μM, with 1% DMSO media serving as the control. After 24 hours for the NP positive groups PMAO-KI NPs were added at a final concentration of 25 μg/mL. After an additional 24 hrs incubation the NPs were irradiated using the previously described protocol at various doses. 24 hours and 72 hours later, depending on the trail, the MTT reagent was added and protocol proceeded as above.

### Iodine Accumulation and Particle Uptake in MCF-7 Cells w/ or w/o tRA.

For iodine uptake study, MCF-7 cells were seeded in 6-well plate (Corning, 3516) at a density of 450,000 cells/mL. 24 hours later the media was replaced, and for tRA positive studies, cell culture medium with a concentration of 1 μM all trans-retinoic acid was added for 24 additional hours. The medium was replaced with a fresh cell medium containing either 0, 12, 25, or 50 μg/mL PMAO-KI NPs. X-ray therapy followed a similar protocol as the MTT experiment. Cells were trypsinized and lysed, and iodine quantified through ICP-MS (Center for Applied Isotope Studies, University of Georgia). For particle uptake studies, MCF-7 cells were seeded in a 8-well Nunc™ Lab-Tek™ II Chamber Slide™ System (Thermo Fisher, 154534PK) at the same density as the iodine ICP study. The PMAO-KI NPs were functionalized with Cy5 dye and the cell nuclei stained with DAPI. Images were taken using a Brightfield Microscope (OLYMPUS TH4-100, Japan). NP uptake quantification was analyzed based on the Cy5 channel fluorescence intensities in the images.

### Clonogenic Assay.

MCF-7 cells were seeded into 6 well plates at a density of either 500,000 cells/mL depending on treatment. After seeding the cells were incubated for 24 hrs before NIS induction as in the MTT assay for the tRA positive groups. 24 hours after tRA addition NPs were added a concentration 50 μg/mL for the NP therapy groups and incubated for an additional 24 hrs. The medium was then replaced, and the groups were irradiated at 0, 2, 4, or 6 Gy before being plated at a cellular density of 500, 1000, 10,000, 50,000, or 500,000 cells/mL respectively. Colonies were stained with crystal violet and CFU/cells were then calculated after about 12 days. These groups were performed in triplicate. Efficacy was calculated through

$$Plating Efficiency = \frac{\# of colonies formed}{\# of cells seeded} \times 100 \% ,$$

$$Survival Fraction = \frac{\# of colonies formed after irradiation}{\# of cells seeded \times P E} .$$

The results were fitted into a linear-quadratic equation (S(D)/S(0)=exp−(aD+bD²)), where SF(D)=Survival Fraction at dose D,

$$[ 1 - {( 1 - {e}^{\frac{- D}{{D}_{0}}} )}^{n} ] ,$$

n=1 for a single dose, D₀=the dose to reduce cell survival to 37% of its value at any point on the final near exponential part of the curve. Dose modification radio at 10% survival

$$D M {R}_{10 \%} = \frac{{D}_{10 \% , Cont}}{{D}_{10 \% , NPs}} ,$$

where D_{10%}=Does required for 10% survival.

### Lipid Peroxidation.

Lipid peroxidation was measured using an Image-iT™ Lipid Peroxidation Kit (Invitrogen, C10445) for live cell analysis. MCF-7 cells were preincubated with 1 μM tRA for tRA positive cells and peroxidation was measured at 5 Gy with 25 μg/mL PMAO-KI NPs for NP positive cells. All the experiments were performed following manufacturer instructions. The ratio of green absorbance to red absorbance measured using SynergyMx 510 nm/590 nm and compared to pre X-ray controls to indicate induced lipid peroxidation.

### γH2Ax Assay.

The DNA damage was studied using anti-γH2AX (Alexa 647) antibody (Millipore Sigma, 07-164-AF647). MCF-7 cells were seeded into 6-well plates at a density of either 500,000 cells/mL and incubated overnight. Cells were treated with 1 μM tRA for tRA positive groups. PMAO-KI NPs were added a concentration 50 μg/mL for the NP therapy groups and incubated for an additional 24 hrs. X-ray radiation at 6 Gy was delivered. Cells were incubated for another 24 h at 37° C. The cells were collected, fixed, permeabilized, and stained with anti-γH2AX antibody according to the protocol from the manufacturer. The presence of γH2AX protein was analyzed using a Millipore Sigma Image Stream Mark II, and both single cell image and statistical data was collected to evaluate DNA damage efficacy.

### Statistical Analysis.

Comparison of multiple assays was performed using a one-way ANOVA followed by a Turkey test. Comparisons of only two groups was performed using a paired t-test Significance was set at p<0.05 represented by * in graphs with ** and *** representing p<0.01 and p<0.005 respectively. All experiments were performed with at least three replicates unless specified. All the data is represented as mean±SEM.

### Results

KI NPs were tested for the ability to enhance radical production under beam irradiation. This was analyzed by subjecting PMAO-KI NP solutions to X-rays (5 Gy) and measuring radicals produced using chemical sensors including dihydroethidium (DHE), singlet oxygen sensor green (SOSG), and aminophenyl fluorescein (APF). These fluorogenic probes are selectively responsive to superoxide, singlet oxygen, and hydroxyl radicals, respectively. Hydroxyl radical, which is the most relevant in RT, showed a remarkable and radiation dose dependent increase (FIG. 2F). Superoxide and singlet oxygen levels, on the other hand, were not significantly elevated with PMAO-KI NPs (FIG. 2G-2I).

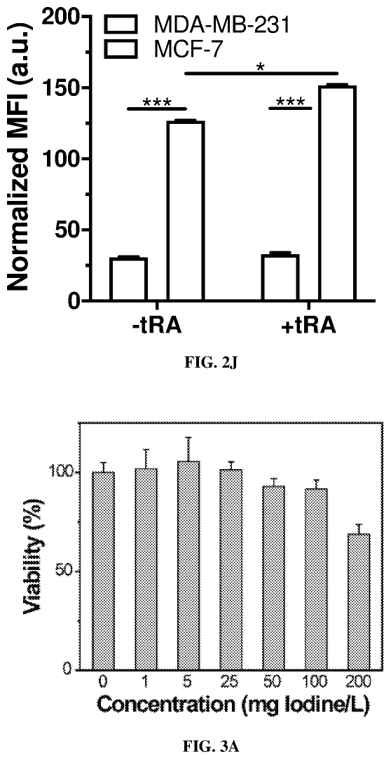

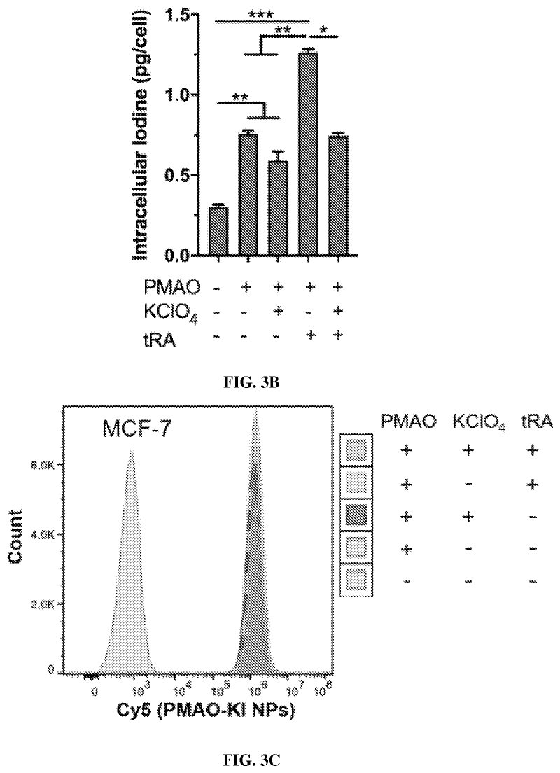

PMAO-KI NPs were tested in vitro with MCF-7 cells, which are NIS positive (FIG. 2J). There was no significant cytotoxicity when the concentration of PMAO-KI NPs was below 100 μg iodine/mL (FIG. 3A). Next, MCF-7 cells were incubated with PMAO-KI NPs at 25 μg/mL for 24 h, and measured iodine contents in cells by inductively coupled plasma mass spectrometry (ICP-MS). Trans retinoic acid (tRA, 1 μM) (Tazebay, et al., Nature medicine, 6 (8), 871 (2000), Kogai, et al., Proceedings of the National Academy of Sciences, 97 (15), 8519-8524 (2000)) was added into the incubation medium to promotes NIS expression (FIG. 3B) (Wapnir, et al., The Journal of Clinical Endocrinology & Metabolism, 88 (4), 1880-1888 (2003), Kogai, et al., Proceedings of the National Academy of Sciences, 97 (15), 8519-8524 (2000)). For comparison, cells treated with KClO₄, an NIS inhibitor, were also tested. Relative to untreated MCF-7 cells, incubation with PMAO-KI NPs doubled the intracellular iodine levels. The iodine uptake was further elevated when tRA was applied and reduced in the presence of KClO₄. Meanwhile, fluorescence microscopy and flow cytometry (FIG. 3C) and found insignificant difference in cellular uptake of Cy5-labeled PMAO-KI NPs between cells treated or not treated with tRA. These results indicate that a significant portion of iodine enters cells in the form of free iodide rather than intact NPs.

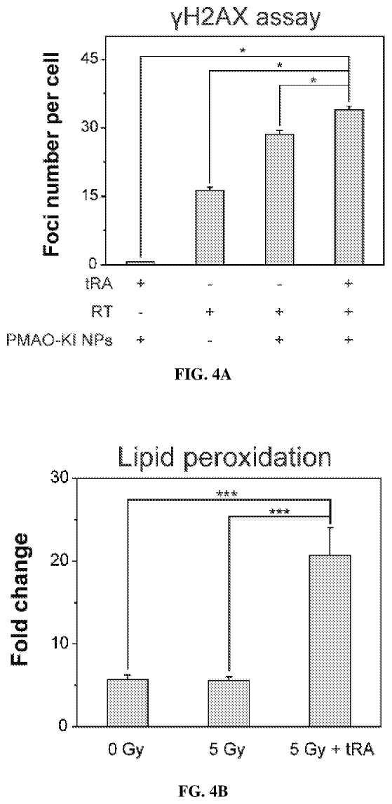

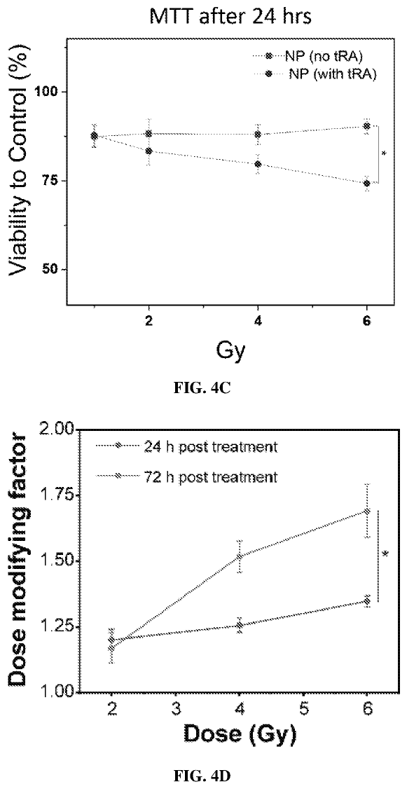

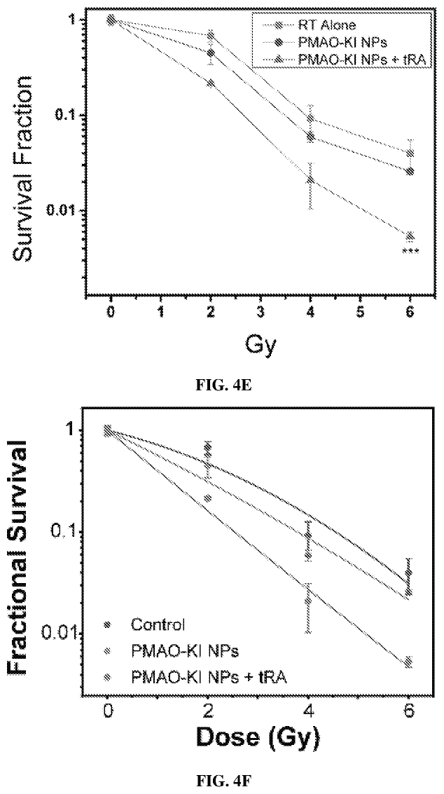

RT-induced DNA damage by anti-γH2AX staining was also examined. Relative to radiation alone (RT, 5 Gy), radiation in the presence of PMAO-KI NPs increased γH2AX foci number from 17.5 to 28.7 foci/cell. This number was increased further to 32.8 foci/cell when tRA was added (FIG. 4A). Meanwhile, PMAO-KI NPs plus RT in the presence of tRA elevated lipid peroxidation level by 20.72%, compared to 5.68% for RT alone (FIG. 4B). The increased DNA damage and lipoid peroxidation is attributed to an enhanced production of hydroxyl radical as a result of iodide accumulation in cells. Notably, previous studies show that iodide can accumulate in the lipid membrane through physical interaction, (Arroyo-Helguera, et al., Endocrine-related cancer, 13 (4), 1147-1158 (2006)) which may contributes to enhanced lipid peroxidation.

The impact of PMAO-KI NPs on cell viability under radiation was assessed by MTT assays. In the presence of tRA, PMAO-KI NPs plus RT (5 Gy) reduced the 24-h cell viability by 34.66% relative to treatment without tRA (FIG. 4C). The benefits of PMAO-KI NPs were also confirmed by a dose-dependent decrease of cell viability at both 24 hrs and 72 hrs (FIG. 4D). The radiosensitizing effects of PMAO-KI NPs were further analyzed by clonogenic assays. Compared to RT alone, PMAO-KI NPs plus RT significantly reduced colony formation at all tested doses (FIG. 4E). The survival fraction was 0.68±0.10 for RT alone at 2 Gy, which was reduced to 0.22±0.01 for PMAO-KI NPs plus RT. At 6 Gy, the survival fraction was only 0.0053±0.0006 for PMAO-KI NPs plus RT, compared to 0.040±0.015 for RT alone (FIG. 4E). When fitting the results into the linear-quadratic model (S(D)/S(0)=exp−(aD+bD²)), it was determined that the dose required for 10% survival (D₁₀) was 4.6126 for RT, 3.9399 for PMAO-KI NPs plus RT, and 2.6006 for PMAO-KI NPs plus RT in the presence of tRA (FIG. 4F); this represents a remarkable dose modifying radio (DMR) of 1.7737 for the PMAO-KI NPs plus tRA combination.

| Group | a | b | D₁₀ | DMF |
|---|---|---|---|---|
| RT only | 0.0274 | 0.1023 | 4.6126 | — |
| PMAO-KI NPs | 0.4136 | 0.0433 | 3.9399 | 1.1707 |
| PMAO-KI NPs + tRA | 0.8814 | 0.0016 | 2.6006 | 1.7737 |

### Example 3: KI NPs can Enhance Radiotherapy of Tumors In Vitro

### Materials and Methods

### Animals and Tumor Inoculation.

Female nude mice (4-5 weeks old, 20) were obtained from (Charles River, USA). All experiments were conducted in accordance with the guidelines from the University of Georgia institutional animal care and use committee (Animal Use Protocol Number: A2017 11-002-A10). One week after mice arrival drinking water was supplemented with 8 mg/L estradiol for MCF-7 tumor model establishment. After one week of pre-supplementation estradiol, 5 million MCF-7 cells in the mixture of 150 μL Matrigel® Matrix (Corning, 354234) and PBS at 1:1 ratio were inoculated into the right flank of mice.

### Preparation of Radioactive Iodide Nanoparticles

### Synthesis of ¹³¹I-KI NPs.

3.9 mCi ¹³¹I-NaI aqueous solution (40 μL) was dried in an Eppendorf tube (1.5 mL) on a thermal mixer at 95° C. over 2 hours before it was cooled down and 0.2 ml hexane was added into the tube to suspend the ¹³¹I-NaI. At the same time, potassium acetate (42.45 mg) was added into a glass tube vial (10 mL) with an aluminum screw cap and a stir bar inside before EtOH (1.25 ml) was injected and stirred until clear solution formed. Then hexane (1.05 ml) was added into the tube vial and stirred until the solution became clear. Sonicate it if necessary. Oleyamine (0.125 ml, it was heated to be clear and then cooled down to room temperature) was then added into the tube vial and stirred. Acetyl iodide (32 ul) was slowly added into the 0.2 ml ¹³¹I solution in the Eppendorf tube before the mixed iodide was slowly transferred into the tube vial. And it was stirred at room temperature for another 30 minutes. The final suspension was a yellow solution with white precipitate. It was separated into aliquots (800 uL×3) in Eppendorf tubes and centrifuged for 10 minutes at 10,000 rpm. Finally, the brown supernatants were discarded and white precipitates were rinsed with hexanes (100 uL×3) before the white particles were completely dried on a thermal mixer at 100° C. for 15 minutes. The nanoparticles were cool to room temperature and activities were determined with a dose calibrator. Over a 3-hour synthetic process, 1.6 mCi ¹³¹I-KI NPs were obtained as the final product, 600 uCi of which was further suspended into PBS buffer (200 uL), 20 μL (60 uCi) of which was then directly injected into a mice tumor.

### Synthesis of ¹²⁴I-KI NPs.

The ¹²⁴I-KI NPs were synthesized following the same protocol as the ¹³¹I-KI NPs described above. 2.1 mCi ¹²⁴I-NaI was used and 830 uCi ¹²⁴I-KI NPs were obtained. 312 uCi was suspended into PBS buffer (200 uL), 20 uL (30 uCi) of which was then directly injected into a mice tumor.

Preparation of ¹³¹I-KI solution and ¹²⁴I-KI solution 6 mg KI was dissolved in the PBS buffer (200 uL) in an Eppendorf tube before 600 uCi ¹³¹I-NaI aqueous solution was added into the buffer. And 20 μL of the mixed buffer solution (60 uCi) was the directly injected into a mice tumor.

6 mg KI was dissolved in the PBS buffer (200 uL) in an Eppendorf tube before 300 uCi ¹²⁴I-NaI aqueous solution was added into the buffer. And 20 μL of the mixed buffer solution (30 uCi) was the directly injected into a mice tumor.

### Therapy Studies.

MCF-7 tumor bearing mice were randomly divided into 4 groups (n=5 for each group), including 1) PBS, 2) PMAO-KI NPs, 3) Radiation (RT) at 5 Gy, 4) PMAO-KI NPs+RT (5 Gy). Therapy was started once tumor volume reaches 100 mm³. tRA was intratumarally injected at 1 μM in 50 uL PBS on Day 0, 3 and 5 to stimulate uniform NIS expression. PMAO-KI NPs were intratumarally injected at 50 μg/mL in 50 μL PBS on Day 4. The tumor size and body weight were inspected every 3 days. The tumor was measured in two dimensions with a caliper, and the tumor volume was estimated as (length)×(width)²/2. RT Estradiol supplementation was continued throughout the endpoints. At the end of the therapy experiment, autopsies were performed. The tumor and major organs were dissected for morphological and histological examination. In particular, tissues in all groups were fixed in 10% (v/v) paraformaldehyde. These tissues were then sectioned into 4 μm slices for H&E, Ki-67 staining to evaluate cell death and proliferation, respectively. Images were acquired using a light microscope (OLYMPUS TH4-100, Japan).

### Results

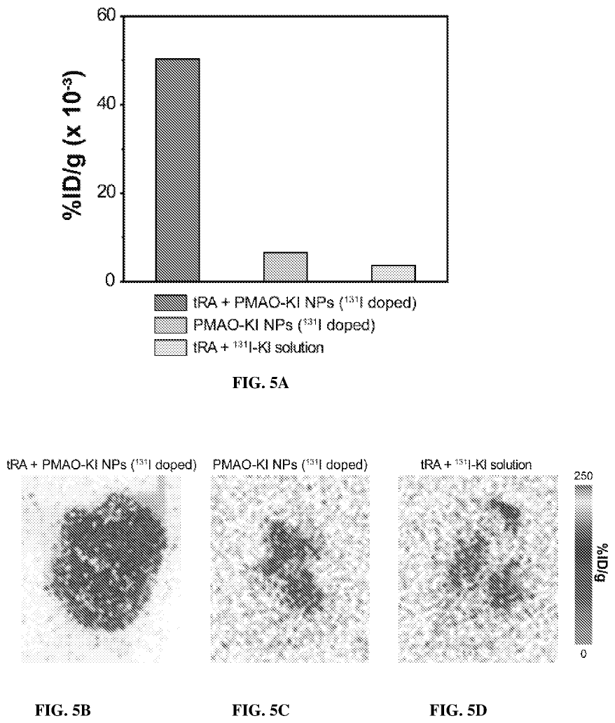

Tumor retention of iodine was evaluated in vivo in MCF-7 tumor bearing mice. To this end, ¹³¹I-doped PMAO-KI NPs (¹³¹I-PMAO-KI NPs) were synthesized, and intratumorally (i.t.) injected (˜4.6 MBq) into animals (n=3). tRA was pre-administered into tumors to promote NIS expression (Kogai, et al., Proceedings of the National Academy of Sciences, 97 (15), 8519-8524 (2000)). For comparison, ¹³¹I-KI solution at the same dosimetry plus tRA or ¹³¹I-PMAO-KI NPs alone (i.e. without tRA) were i.t. injected. After 72 hrs, the animals were euthanized, and tumors and major organs were harvested and subjected to gamma counting to evaluate iodine bio-distribution. Relative to ¹³¹I-PMAO-KI NPs only, ¹³¹I-PMAO-KI NPs plus tRA showed a significant increase of iodine retention in tumors (FIGS. 5A-5D), which agrees well with what was observed in vitro. Radioactivity in tumors treated with ¹³¹I-PMAO-KI NPs plus tRA was also high relative to those treated with ¹³¹I-KI solution plus tRA (FIGS. 5A-5D), suggesting that controlled release may also contribute to improved tumor retention. This postulation is supported by the observation that overall radioactivity at 72 hrs is high in the ¹³¹I-PMAO-KI NPs+tRA group relative to the control groups (FIG. 5E). Notably, in addition to tumors, significant radioactivities were found in the thyroid and stomach, where NIS expression is also high (Harun-Or-Rashid, et al., Thyroid Res, 3 (1), 3 (2010)).

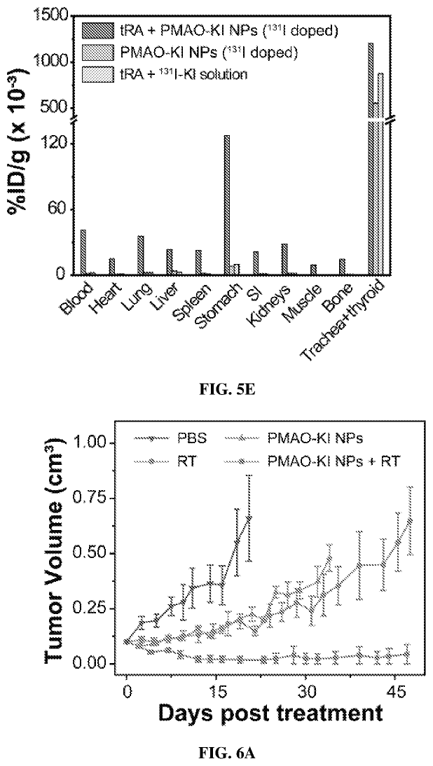

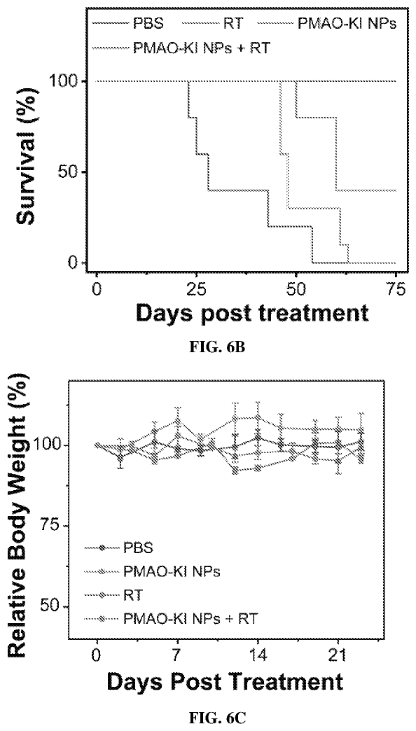

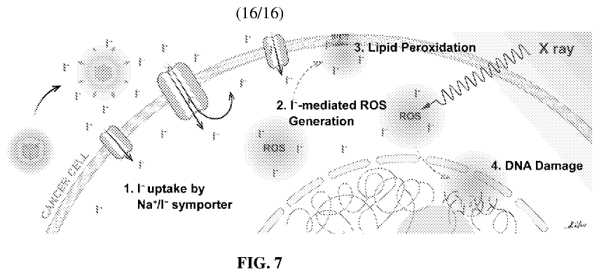

Lastly, treatment efficacy in MCF-7 tumor bearing mice was examined. Briefly, PMAO-KI NPs (50 μg iodine/mL, 50 μL) were i.t. injected, followed by radiation (5 Gy) applied to tumors. The rest of the animal body was protected by lead (n=5). For comparison, PBS, RT alone and PMAO-KI NPs alone were investigated. tRA was pre-administered into all animals to promote NIS expression. Animals in the PBS and PMAO-KI NPs groups showed rapid tumor progression (FIG. 6A), resulting in a relatively short animal survival (32 and 50 days on average, respectively). RT only caused a moderate tumor suppression and a slightly extended animal survival (57 days, FIG. 6B). However, 60% of the animals in the RT group had either died or met a humane endpoint by day 75. As a comparison, all animals in the PMAO-KI NPs plus RT (PMAO-KI NPs+RT) group experienced significant tumor regression. Remarkably, 80% of the tumors became impalpable after two weeks, and 60% of the animals remained tumor-free after 60 days. All animals in the PMAO-KI NPs+RT group remained alive at the end of the study on Day 75 (FIG. 6B). No mice in the group showed acute toxicity or significant body weight drop throughout the experiment (FIG. 6C).

Post-mortem histology was performed to validate treatment efficacy. H&E staining found large areas of nuclear shrinkage and cell death in the PMAO-KI NPs+RT group. Ki-67 staining also confirmed that the combination led to a significant reduction in cancer cell proliferation compared to PBS, RT alone and PMAO-KI NPs alone. Meanwhile, H&E staining found no signs of adverse effects in major organ tissues, including the brain, heart, kidney, liver, lung and spleen.

PMAO-KI NPs were fabricated and their role as a radiosensitizer for RT against breast cancer was investigated. Remarkable improvement in tumor suppression was observed among animals receiving i.t. injection of PMAO-KI NPs before irradiation. As discussed above, iodine-containing molecules such as Iomeprol have short circulation half-lives and low tumor uptake. NP-enabled controlled release of iodide and NIS-mediated cell uptake of the ion is exploited in the disclosed approach, which provides a unique solution to these issues. While iodine molecule (I₂) shows anti-proliferative effects (Rösner, et al., Oncology letters, 12 (3), 2159-2162 (2016), Arroyo-Helguera, et al., Endocrine-related cancer, 15 (4), 1003-1011 (2008), little toxicity with iodide was observed (FIG. 3A), which agrees with others' observation (Arroyo-Helguera, et al., Endocrine-related cancer, 13 (4), 1147-1158 (2006)). The tumor suppression was attributed to iodide-based enhancement in RT-induced production of radicals and damage to the cellular components, not direct cancer cell killing by iodide.

In the clinic, breast cancer patients after mastectomy or lumpectomy often receive multi-session fractionated RT to prevent recurrence. It is envisioned, for example, that KI NPs can be injected into the tumor bed for sustained iodide release and radiosensitization. The current PMAO-KI formulation has t_{1/2} of ˜24 h. It is possible to increase the coating thickness and/or degree of crosslinking to extend iodide release so that one injection can benefit multiple RT sessions. It is also possible to inject PMAO-KI NPs systemically and rely on either passive or active targeting of NPs for tumor accumulation (Golombek, et al., Adv Drug Deliv Rev, 130, 17-38 (2018)). Radioisotope such as ¹³¹I and ¹²⁵I can be incorporated into PMAO-KI NPs, producing “hot” NPs that function as unique a radiopharmaceutical or a brachytherapy agent for cancer treatment. For these applications, PMAO-KI NPs will play an important role for mitigating radioactivity accumulation in the thyroid and stomach and toxicity to the tissues.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A nanoparticle formed from iodide and ions of an alkali metal or alkaline earth metal.

2. The nanoparticle of claim 1, wherein the alkali metal is selected from potassium, lithium, sodium, rubidium, and cesium, and the alkaline earth metal is selected from magnesium and calcium.

3. (canceled)

4. A nanoparticle formed from iodide and potassium ions.

5. The nanoparticle of claim 4, comprising a radioisotope.

6. The nanoparticle of claim 5, wherein the radioisotope is ¹³¹I, ¹²⁵I, ¹²⁴I, or ¹²³I.

7. The nanoparticle of claim 6, wherein the iodine is, or is formed from, the ¹³¹I, ¹²⁵I, ¹²⁴I, or ¹²³I.

8.-10. (canceled)

11. The nanoparticle of claim 4, further comprising a coating.

12. (canceled)

13. The nanoparticle of claim 11, wherein the coating comprises poly(maleic anhydride alt-1-octadecene).

14.-15. (canceled)

16. The nanoparticle of claim 11, comprising a targeting moiety.

17.-19. (canceled)

20. The nanoparticle of claim 16, wherein the targeting moiety targets the sodium/iodide symporter (NIS).

21.-22. (canceled)

23. The nanoparticle of claim 11 comprising an active agent.

24.-25. (canceled)

26. The nanoparticle of claim 23, wherein the active agent is incorporated into the coating, optionally wherein the active agent is conjugated to a component of the coating.

27. A pharmaceutical composition comprising a plurality of the nanoparticles of claim 4.

28.-29. (canceled)

30. A method of making potassium iodide nanoparticles comprising reacting potassium oleate with I₂ in octadecene in the presence of oleylamine.

31.-32. (canceled)

33. A method of sensitizing a subject to radiation therapy comprising administering to a subject in need thereof an effective amount of the pharmaceutical composition of claim 27.

34. A method of treating a subject for cancer comprising sensitizing the subject to radiation therapy according to method of claim 33 and administering the subject one or more doses of radiation therapy.

35. The method of claim 34, wherein the radiation therapy is ionizing radiation therapy, phototherapy, or proton therapy.

36.-43. (canceled)

44. A method of treating a subject for cancer comprising administering the subject an effective amount of the pharmaceutical composition of claim 27, wherein the nanoparticle comprises a radioisotope or an anti-cancer active agent.

45. A method of imaging a subject in need thereof comprising administering the subject an effective amount of the pharmaceutical composition of claim 27, wherein the nanoparticle comprises a radioisotope, and detecting the radioisotope, optionally wherein the subject has cancer.

46.-51. (canceled)

52. The method of claim 44, wherein cells of the cancer express the Na⁺/I⁻ symporter (NIS).

53.-60. (canceled)
